(19) United States
(12) Patent Application Publication
(10) Pub. No.: US 2020/0353070 A1
(43) Pub. Date: Nov. 12, 2020

(54) SELECTIVE CD8-POSITIVE T CELL-INDUCING VACCINE ANTIGEN

(71) Applicants: Japan as represented by the Director-General of National Institute of Infectious Diseases, Tokyo (JP); ID Pharma Co., Ltd., Tokyo (JP)

(72) Inventors: Tetsuro MATANO, Tokyo (JP); Hiroshi ISHII, Tokyo (JP); Makoto INOUE, Tokyo (JP); Takashi HIRONAKA, Tokyo (JP); Tsugumine SHU, Tokyo (JP); Toyotaka MORI, Tokyo (JP)

(21) Appl. No.: 16/962,579

(22) PCT Filed: Jan. 21, 2019
(86) PCT No.: PCT/JP2019/001607

§ 371 Date: Jul. 16, 2020

(30) Foreign Application Priority Data
Jan. 22, 2018 (JP) 2018-008255

(51) Int. Cl. A61K 39/21; C07K 14/155; C12N 15/86

(52) U.S. Cl. CPC A61K 39/21; A61K 38/00; C12N 15/86; C07K 14/155

(57) ABSTRACT

The present invention provides polypeptides for selectively inducing target antigen-specific CD8-positive T-cell responses. Since induction of human immunodeficiency virus (HIV)-specific CD4-positive T-cell responses by vaccine could promote HIV infection, an HIV vaccine antigen that selectively induces HIV-specific CD8-positive T-cell responses would be useful if obtained. Thus, in the present invention, polypeptide antigens were designed in which 8- to 12-residue amino acid sequences divided from the amino acid sequence of a target antigen protein were connected in an order different from that of the original amino acid sequence. DNA and viral vector vaccines expressing these antigens were tested by inoculation into monkeys. As a result, they were shown to be able to efficiently induce antigen-specific CD8-positive T-cell responses in a selective manner. The instant antigens may be useful as vaccine antigens that induce CD8-positive T cells in a highly selective manner.

### TECHNICAL FIELD

The present invention relates to polypeptides that selectively induce antigen-specific CD8-positive T-cell responses while keeping antigen-specific CD4-positive T-cell responses at low levels, methods for producing such a polypeptide, vaccines expressing such a polypeptide, and the like. The vaccines of the present invention are particularly useful as anti-HIV vaccines.

### BACKGROUND ART

The number of people infected with human immunodeficiency virus (HIV) exceeds 36 million worldwide, and around 1.8 million people are estimated to be newly infected annually. As such, the spread of HIV infection is a serious problem. The development of an HIV vaccine is an internationally important task to control the spread of HIV infection; however, no effective vaccine has yet been put into practical use. The HIV infection is a fatal infection which, in general, is not cured naturally and develops into chronic persistent infection, leading to acquired immunodeficiency syndrome (AIDS). Treatment with anti-HIV drugs has made it possible to prevent the onset of AIDS, but does not lead to cure because the virus is difficult to eliminate from the body. Therefore, infected persons need to be treated with anti-HIV drugs almost for life (NPL 12). Recently, in addition to the issues of side effects and emergence of drug-resistant virus under long-term medication, high medical costs and acceleration of disorders associated with chronic inflammation such as osteoporosis, cardiovascular disorders, brain and cognitive disorders, and renal disorders, have increasingly become serious problems (NPLs 13-15). However, the number of HIV-infected people is continuously rising in Africa and other parts of the world. In order to control the spread of infection, the development of an effective HIV vaccine is recognized as one of the most internationally important tasks, as well as the promotion of early diagnosis and early treatment. However, an HIV vaccine with established effectiveness has not been developed yet.

Induction of CD8-positive T-cell responses, which are believed to play a central role in suppressing HIV replication, is one of the key strategies in developing HIV vaccines (NPLs 1-4). In developing an HIV vaccine inducing CD8-positive T cells, optimization of antigen delivery and optimization of antigen are considered important. As for the delivery method, a number of vectors capable of efficiently inducing CD8-positive T cells, such as adenoviral vectors (NPL 7), cytomegalovirus vectors (NPL 8), and adenovirus/poxvirus vectors (NPL 9), have been developed.

On the other hand, antigens for inducing effective CD8-positive T cells may need further optimization. CD8-positive T cells specific for a viral antigen specifically recognize 8- to 11-mer peptide fragments (epitopes) derived from the viral antigen that are bound with the major histocompatibility complex (MHC) class I molecule and presented on the surface of virus-infected cells, and damage the infected cells (NPL 16). It is known that what is targeted by CD8-positive T-cell responses depends on the host's MHC class I genotype, and different target viral antigens cause the varying ability of CD8-positive T cells to suppress virus replication (effectiveness) (NPL 17). Moreover, domination of poorly effective CD8-positive T-cell responses results in inhibition of the induction of effective CD8-positive T-cell responses (immunodominance) (NPL 18). Therefore, an antigen needs to be designed so as to induce highly effective CD8-positive T-cell responses selectively. Recent analyses of HIV-infected individuals and simian AIDS models have shown that CD8-positive T-cell responses targeting Gag and Vif antigens have a strong ability to suppress viral replication (NPLs 17-20). The Gag capsid (CA) antigen is also promising as a candidate target region for CD8-positive T cells because of its highly conserved structure (NPL 21).

Conventional HIV vaccine methods induce not only HIV antigen-specific CD8-positive T cells but also HIV antigen-specific CD4-positive T cells at the same time. CD4-positive T cells specific for a viral antigen specifically recognize peptide fragments (epitopes) derived from the viral antigen that are bound with the MHC class II molecule and presented on the surface of antigen-presenting cells, and elicit antigen-specific responses. However, because HIV more preferentially targets HIV antigen-specific CD4-positive T cells to proliferate (NPL 10), vaccine-mediated induction of HIV antigen-specific CD4-positive T cells may lead to acceleration of HIV proliferation. In fact, the analysis of a simian immunodeficiency virus (SIV)-infected simian AIDS model reportedly showed that vaccine-mediated induction of SIV antigen-specific CD4-positive T cells was associated with acceleration of SIV proliferation in the acute phase after SIV exposure (NPL 11). Therefore, achieving antigen optimization requires not only designing a target of effective CD8-positive T cells but also developing a method for inducing effective HIV antigen-specific CD8-positive T cells selectively while suppressing the induction of HIV antigen-specific CD4-positive T cells as much as possible. However, antigen design from this point of view has not been done so far.

### CITATION LIST

### Non-Patent Literature

[NPL 1] Koup R A. et al., J Virol. 68:4650-4655. 1994.
[NPL 2] Matano T. et al., J Virol. 72: 164-169. 1998.
[NPL 3] Schmitz J E. et al., Science. 283: 857-860. 1999.
[NPL 4] Goulder P J. et al., Nat Rev Immunol. 4: 630-640. 2004.
[NPL 5] Matano T. et al., J Exp Med. 199: 1709-1718. 2004.
[NPL 6] Nyombayire J. et al., J Infect Dis. 215: 95-104. 2017.
[NPL 7] Wilson N A. et al., J Virol. 80: 5875-5885. 2006.
[NPL 8] Hansen S G. et al., Nature. 473: 523-527. 2011.
[NPL 9] Barouch D H. et al., Nature. 482: 89-93. 2012.
[NPL 10] Douek D C. et al., Nature. 417: 95-98. 2002.
[NPL 11] Terahara K. et al., J Virol. 88: 14232-14240. 2014.
[NPL 12] Fischer M. et al., AIDS. 17: 195-199. 2003.
[NPL 13] Kirk G D. et al., Clin Infect Dis. 45: 103-110. 2007.
[NPL 14] Hsue P Y. et al., Curr Opin HIV AIDS. 12: 534-539. 2017.
[NPL 15] Khoury G. et al., J Infect Dis. 215: 911-919. 2017.
[NPL 16] Hewitt E W. et al., Immunology. 110: 163-169. 2003.
[NPL 17] Kiepiela P. et al., Nat Med. 13: 46-53. 2007.
[NPL 18] Akram A. et al., Clin Immunol. 143: 99-115. 2012.
[NPL 19] Mudd P A. et al., Nature. 491: 129-133. 2012.
[NPL 20] Iwamoto N. et al., J Virol. 88: 425-433. 2014.
[NPL 21] Goulder P J. et al., Nat Rev Immunol. 8: 619-630. 2008.
[NPL 22] Tsukamoto T. et al., J Virol. 83: 9339-9346. 2009.
[NPL 23] Ishii H. et al., J Virol. 86:738-745. 2012.
[NPL 24] Letourneau S. et al., PLoS One. 2: e984. 2007.
[NPL 25] Mothe B. et al., J Transl Med. 9: 208. 2011.

### SUMMARY OF INVENTION

### Technical Problem

An objective of the present invention is to provide a polypeptide that selectively induces antigen-specific CD8-positive T cells while suppressing the induction of antigen-specific CD4-positive T cells, and a vaccine and the like containing that polypeptide.

### Solution to Problem

As stated above, the induction of CD8-positive T-cell responses, which are believed to play a central role in suppressing HIV replication, is one of the key strategies in developing HIV vaccines, and antigen design is considered important in developing a CD8-positive T cell-inducing HIV vaccine.

Thus, the present inventors contemplated designing an antigen based on a new method in order to selectively induce antigen-specific CD8-positive T cells while suppressing the induction of antigen-specific CD4-positive T cells. Specifically, first, the inventors divided the amino acid sequence of a target antigen protein into partial amino acid sequences having a length that is at least a length typical of MHC class I epitopes but not a length typical of MHC class II epitopes. The inventors then connected the divided partial amino acid sequences so as not to again form many consecutive partial amino acid sequences of the target antigen protein having a typical MHC class II epitope length, for example, by changing the order or placing overlaps and spacers. By doing so, the inventors formed an amino acid sequence containing MHC class I epitopes of the target antigen but not MHC class II epitopes of the target antigen.

Specifically, in the Examples, noting the fact that optimum epitopes for CD4-positive T cells are 13- to 18-mer peptides while those for CD8-positive T cells are 8- to 11-mer peptides, the present inventors designed novel antigens by connecting 11-mer peptides derived from HIV target antigens in tandem in order to induce effective HIV antigen-specific CD8-positive T cells selectively. The antigens were designed by fragmenting the amino acid sequences of viral Gag CA and Vif, which are target regions for effective CD8-positive T cells, into 11-mer peptides, then rearranging these peptides and connecting them in tandem using alanine as spacers (TCT11) (FIG. 1). In a similar manner, a total of 8 tandemly-connected antigens (A to H) were designed, for each of which the starting amino acid position of the peptides in each target antigen region was shifted by one amino acid. All 8 antigens cover all theoretically possible CD8-positive T-cell epitopes present in the target regions. Meanwhile, these antigens do not contain virus-derived 12-mer or longer peptides, and therefore, theoretically, do not contain optimum epitopes for viral antigen-specific CD4-positive T cells. Thus, they should not efficiently induce CD4-positive T cells.

Although the thus designed tandemly-connected antigens correspond to the amino acid sequences of the target antigens in the short range of 11-mer, their whole amino acid sequences are completely different from those of the target antigens. To examine whether these connected antigens can efficiently induce specific CD8-positive T cells against the target antigen proteins, viral vectors expressing the connected antigens were constructed and inoculated into individuals. As a result, the inoculated individuals were found to show a significantly increased frequency of target antigen-specific CD8-positive T cells, but no change in the frequency of target antigen-specific CD4-positive T cells (FIG. 2). Thus, it was demonstrated that a polypeptide produced by rearranging the amino acid sequence of an antigen protein according to the present invention can be used as an antigen to selectively induce immune responses mediated by MHC class I epitopes of the target antigen.

With the method of the present invention, it is not necessary to identify an MHC class I epitope of a target antigen in advance. A polypeptide constructed by rearranging the amino acid sequence according to the method of the present invention can be inoculated as an antigen to selectively induce immune responses mediated by MHC class I epitopes of the target antigen while avoiding the induction of immune responses mediated by MHC class II epitopes of the target antigen. This method is highly versatile and can be used not only for infectious viruses such as HIV but for a wide range of target proteins in general to selectively induce the response of target antigen-specific CD8-positive T cells.

Thus, the present invention relates to antigens that selectively induce antigen-specific CD8-positive T cells while suppressing the induction of antigen-specific CD4-positive T cells, and vaccines and the like including such an antigen. More specifically, the present invention relates to each of the inventions recited in the claims. It should be noted that inventions consisting of any combination of two or more inventions recited in claims that refer to the same claim are also intended herein. Specifically, the present invention relates to the following:

[1] A polypeptide comprising multiple peptides connected together, wherein each of the multiple peptides has an amino acid sequence of eight to twelve residues included in the amino acid sequence of an antigen protein.
[2] The polypeptide of [1], wherein the eight- to twelve-residue peptides are connected in an order different from that in the antigen protein.
[3] The polypeptide of [1] or [2], which does not substantially comprise a partial amino acid sequence of 13 or more consecutive residues in the antigen protein.
[4] The polypeptide of any one of [1] to [3], wherein the amino acid sequences of the multiple peptides optionally comprise an overlap.
[5] The polypeptide of [4], wherein the overlap consists of one to four residues.
[6] The polypeptide of any one of [1] to [5], wherein each of the connection sites optionally comprises a spacer.
[7] The polypeptide of [6], wherein the spacer consists of one to four amino acid residues.
[8] The polypeptide of any one of [1] to [7], wherein at least 20 eight- to twelve-residue peptides are connected together.
[9] A nucleic acid encoding the polypeptide of any one of [1] to [8].
[10] A vector comprising the nucleic acid of [9].
[11] The vector of [10], which is a Sendai virus vector.
[12] A vaccine comprising the polypeptide of any one of [1] to [8], a nucleic acid encoding the polypeptide, or a vector comprising the nucleic acid.
[13] The vaccine of [12], wherein the antigen protein is derived from an antigen protein of a human immunodeficiency virus.
[14] A method for selectively inducing CD8-positive T cells specific for a target antigen, which comprises inoculating the vaccine of [12] or [13].
[15] A method for producing the polypeptide of [1] or a nucleic acid encoding the polypeptide, which comprises:
(i) dividing an amino acid sequence encoding an antigen protein into amino acid sequences of eight to twelve residues, wherein the divided amino acid sequences may or may not overlap with one another;
(ii) connecting the divided amino acid sequences in such a way as not to become the same as the amino acid sequence of the antigen protein, wherein a spacer may or may not be inserted in each of the connection sites of the divided amino acid sequences; and
(iii) obtaining a polypeptide comprising an amino acid sequence resulting from step (ii) or a nucleic acid encoding the polypeptide.

In addition, the present invention also encompasses the following inventions:

[16] The polypeptide of any one of [1] to [8], wherein the multiple peptides are connected together via a spacer.
[17] The polypeptide of any one of [1] to [8] and [16], wherein 10 or more eight- to twelve-residue amino acid sequences are connected together.
[18] The polypeptide of [17], wherein 20 or more eight- to twelve-residue amino acid sequences are connected together.
[19] The polypeptide of [17], wherein 30 or more 8- to 12-residue amino acid sequences are connected together.
[20] The polypeptide of any one of [1] to [8] and [16] to [19], wherein the amino acid sequence of 8 to 12 residues or less is an amino acid sequence of 8 to 11 residues.
[21] The polypeptide of any one of [1] to [8] and [16] to [20], wherein the total number of residues of a partial amino sequence of consecutive 13 or more residues of the antigen protein is 20% or less of the total number of residues of the connected amino acid sequences.
[22] The polypeptide of [21], wherein the total number of residues of a partial amino sequence of consecutive 13 or more residues of the antigen protein is 10% or less or 5% or less of the total number of residues of the connected amino acid sequences.

Furthermore, the present invention also encompasses the following inventions:

[23] The polypeptide of any one of [1] to [8], which comprises an amino acid sequence in which multiple amino acid sequences of 12 residues or less selected from the amino acid sequence of the antigen protein are connected together.
[24] The polypeptide of [23], wherein the multiple amino acid sequences are connected so as not to become the same amino acid sequence of the antigen protein.
[25] The polypeptide of [23] or [24], wherein the multiple amino acid sequences are connected so as not to substantially generate an amino acid sequence of consecutive 13 or more residues of the antigen protein.
[26] The polypeptide of [23] or [24], wherein the multiple amino acid sequences are connected such that the number of connections generating an amino acid sequence of consecutive 13 or more residues of the antigen protein is 20% or less, 10% or less, or 5% or less of the total number of connections.
[27] The polypeptide of [26], wherein multiple amino acid sequences of 11 residues or less selected from the amino acid sequence of the antigen protein are connected together, wherein the multiple amino acid sequences are connected so as not to substantially generate an amino acid sequence of consecutive 12 or more residues of the antigen protein, or such that the number of connections generating an amino acid sequence of consecutive 12 or more residues of the antigen protein is 20% or less, 10% or less, or 5% or less of the total number of connections.
[28] The polypeptide of any one of [23] to [27], wherein the multiple peptides are connected together via a spacer.
[29] The polypeptide of [28], wherein the spacer consists of 1 to 4 amino acid residues.
[30] The polypeptide of any one of [20] to [29], wherein the divided amino acid sequences overlap one another.
[31] The polypeptide of [30], wherein the divided amino acid sequences overlap by 1 to 4 residues.
[32] The polypeptide of any one of [23] to [31], wherein at least 10 or more divided amino acid sequences are connected together.
[33] The polypeptide of [32], wherein 20 or more divided amino acid sequences are connected together.
[34] The polypeptide of [32], wherein 30 or more divided amino acid sequences are connected together.
[35] The polypeptide of any one of [23] to [34], wherein the amino acid sequence of the antigen protein is divided into amino acid sequences of 5 to 12 residues.
[36] The polypeptide of any one of [23] to [34], wherein the amino acid sequence of the antigen protein is divided into amino acid sequences of 8 to 12 residues.
[37] The polypeptide of any one of [23] to [34], wherein the amino acid sequence of the antigen protein is divided into amino acid sequences of 8 to 11 residues.

Furthermore, the present invention also encompasses the following inventions:

[38] A nucleic acid encoding the polypeptide of any one of [16] to [37].
[39] A vector comprising the nucleic acid of [38].
[40] The vector of [39], which is a Sendai virus vector.
[41] A vaccine comprising the polypeptide of any one of [16] to [37], a nucleic acid encoding the polypeptide, or a vector comprising the nucleic acid.
[42] The vaccine of [41], wherein the antigen protein is derived from an antigen protein of a human immunodeficiency virus.
[43] A method for selectively inducing CD8-positive T cells specific for a target antigen, which comprises inoculating the vaccine of [41] or [42].

Furthermore, the present invention also encompasses the following inventions:

[44] Use of the polypeptide of any one of [1] to [8] and [16] to [37], a nucleic acid encoding the polypeptide, or a vector comprising the nucleic acid, for selectively inducing target antigen-specific CD8-positive T cells.
[45] Use of the polypeptide of any one of [1] to [8] and [16] to [37], a nucleic acid encoding the polypeptide, or a vector comprising the nucleic acid, for manufacture of a medicament or an agent for selectively inducing target antigen-specific CD8-positive T cells.

Furthermore, the present invention also encompasses the following inventions:

[46] A method of vaccination comprising inoculating the polypeptide of any one of [1] to [8] and [16] to [37], a nucleic acid encoding the polypeptide, or a vector comprising the nucleic acid.
[47] The method of [46], wherein a plurality of the polypeptides of any one of [1] to [8] and [16] to [37], a plurality of nucleic acids encoding the polypeptides, or a plurality of vectors comprising the nucleic acids, are administered.
[48] The method of [46] or [47], which comprises further inoculating an additional polypeptide that is not the polypeptide of any one of [1] to [8] and [16] to [37], a nucleic acid encoding the additional polypeptide, or a vector comprising the nucleic acid.
[49] The method of [48], wherein the additional polypeptide that is not the polypeptide of any one of [1] to [8] and [16] to [37], the nucleic acid encoding the polypeptide, or the vector comprising the nucleic acid, is inoculated first.
[50] The method of [48] or [49], wherein the additional polypeptide is the antigen protein or a partial peptide thereof.
[51] The method of any one of [48] to [50], wherein the vector comprising the nucleic acid encoding the additional polypeptide is a DNA vector.
[52] Use of the polypeptide of any one of [1] to [8] and [16] to [37], a nucleic acid encoding the polypeptide, or a vector comprising the nucleic acid, for vaccination.
[53] The use of [52], which is for administering a plurality of the polypeptides of any one of [1] to [8] and [16] to [37], a plurality of nucleic acids encoding the polypeptides, or a plurality of vectors comprising the nucleic acids.
[54] The use of [52] or [53], wherein an additional polypeptide that is not the polypeptide of any one of [1] to [8] and [16] to [37], a nucleic acid encoding the additional polypeptide, or a vector comprising the nucleic acid, is further inoculated.
[55] The method of [54], wherein the additional polypeptide that is not the polypeptide of any one of [1] to [8] and [16] to [37], the nucleic acid encoding the polypeptide, or the vector comprising the nucleic acid, is inoculated first.
[56] The use of [54] or [55], wherein the additional polypeptide is the antigen protein or a partial peptide thereof.
[57] The method of any one of [54] to [56], wherein the vector comprising the nucleic acid encoding the additional polypeptide is a DNA vector.
[58] Use of the polypeptide of any one of [1] to [8] and [16] to [37], a nucleic acid encoding the polypeptide, or a vector comprising the nucleic acid, for manufacture of a medicament or an agent for vaccination.
[59] The use of [58], which is for administering a plurality of the polypeptides of any one of [1] to [8] and [16] to [37], a plurality of nucleic acids encoding the polypeptides, or a plurality of vectors comprising the nucleic acids.
[60] The use of [58] or [59], wherein an additional polypeptide that is not the polypeptide of any one of [1] to [8] and [16] to [37], a nucleic acid encoding the additional polypeptide, or a vector comprising the nucleic acid, is further inoculated.
[61] The method of [60], wherein the additional polypeptide that is not the polypeptide of any one of [1] to [8] and [16] to [37], the nucleic acid encoding the polypeptide, or the vector comprising the nucleic acid, is inoculated first.
[62] The use of [60] or [61], wherein the additional polypeptide is the antigen protein or a partial peptide thereof.
[63] The method of any one of [60] to [62], wherein the vector comprising the nucleic acid encoding the additional polypeptide is a DNA vector.
[64] Use of the polypeptide of any one of [1] to [8] and [16] to [37], a nucleic acid encoding the polypeptide, or a vector comprising the nucleic acid, for vaccination.
[65] The use of [64], which is for administering a plurality of the polypeptides of any one of [1] to [8] and [16] to [37], a plurality of nucleic acids encoding the polypeptides, or a plurality of vectors comprising the nucleic acids.
[66] The use of [64] or [65], wherein an additional polypeptide that is not the polypeptide of any one of [1] to [8] and [16] to [37], a nucleic acid encoding the additional polypeptide, or a vector comprising the nucleic acid, is further inoculated.
[67] The method of [66], wherein the additional polypeptide that is not the polypeptide of any one of [1] to [8] and [16] to [37], the nucleic acid encoding the polypeptide, or the vector comprising the nucleic acid, is inoculated first.
[68] The use of [66] or [67], wherein the additional polypeptide is the antigen protein or a partial peptide thereof.
[69] The method of any one of [66] to [68], wherein the vector comprising the nucleic acid encoding the additional polypeptide is a DNA vector.

It should be noted that any technical matter or any combination of technical matters described in the present specification are intended herein. In addition, inventions that correspond to those inventions except that any matter or any combination of matters described in the present specification are excluded are also intended herein. Moreover, a specific embodiment described herein in relation to the present invention is meant to disclose not only that embodiment but also an invention corresponding to a more generic invention disclosed herein including that embodiment from which that embodiment is excluded.

### Effects of the Invention

As stated above, the present invention is useful for selectively inducing CD8-positive T cells for a desired antigen. For example, the present invention enables an AIDS vaccine to induce effective HIV antigen-specific CD8-positive T cells selectively while suppressing the induction of HIV antigen-specific CD4-positive T cells as much as possible.

### BRIEF DESCRIPTION OF DRAWINGS

FIG. 1 shows a summary of target 11-mer connected antigen TCT11.

FIG. 2 shows an inoculation test of SCaV11 antigen-expressing vaccines in the chronic phase of SIV replication-controlled monkeys.

FIG. 3 shows an inoculation test of SCaV11 antigen-expressing vaccines in non-infected monkeys.

FIG. 4 shows antigen-specific T-cell responses after vaccination as in FIG. 3.

FIG. 5 shows antigen-specific T-cell responses in the lymph node after vaccination.

### DESCRIPTION OF EMBODIMENTS

The embodiments of the present invention are specifically described below.

In the present invention, a “vaccine” refers to a composition for eliciting immune responses against an antigen. For example, it refers to a composition used for preventing or treating a contagious disease, an infection, and the like. A vaccine contains an antigen or can express an antigen, by which it can induce immune responses against the antigen. The polypeptides of the present invention, and nucleic acids and vectors encoding the polypeptides, are useful as vaccines for preventing or treating the infection, transmission, and epidemic of pathogenic microorganisms. The vaccines can be used in any form as desired.

An “antigen” refers to, in general, a molecule that contains one or more epitopes (portions of antigen recognized by antibodies or immune cells) and may stimulate the host immune system and induce antigen-specific immune responses. The immune responses may be humoral immune responses and/or cellular immune responses. In the present invention, the epitopes include not only epitopes formed from primary structures but also epitopes depending on protein conformations. The “antigen” is also referred to as “immunogen”.

In the present invention, a “viral vector” is a vector having genomic nucleic acid derived from a virus, and capable of expressing a transgene incorporated into the nucleic acid after being introduced into cells. For example, paramyxovirus vectors are chromosomally non-integrating viral vectors and expressed within the cytosol. Therefore, they have no risk of integrating a transgene into host chromosomes (nuclear chromosomes). They are therefore highly safe, and can also be removed from infected cells. In the present invention, paramyxovirus vectors include infectious viral particles, and also include viral cores, complexes composed of a viral genome and viral proteins or complexes composed of a non-infectious viral particle and such that are capable of expressing a gene they carry when introduced into cells. For example, in the paramyxovirus, the ribonucleoprotein (viral core) composed of a paramyxovirus genome and paramyxovirus proteins binding to it (NP, P, and L proteins) can express a transgene intracellularly when introduced into cells (WO00/70055). The introduction into cells may be performed using a transfection agent and such, as appropriate. Such ribonucleoproteins (RNPs) are also included in paramyxovirus vectors in the present invention. Preferably, a paramyxovirus vector in the present invention is a particle in which the aforementioned RNP is enclosed by a biological membrane derived from the cell membrane.

The present invention provides polypeptides useful for selectively inducing CD8-positive T cells specific for a target antigen protein while suppressing the induction of CD4-positive T cells specific for the target. Such a polypeptide contains an amino acid sequence in which partial amino acid sequences excised from an antigen protein such that they have a length that is at least a length typical of MHC class I epitopes but not a length typical of MHC class II epitopes are connected together in such a way as not to become the same as the original amino acid sequence of the antigen protein (i.e. the amino acid sequence of the antigen protein). Here, the length typical of MHC class I or MHC class II epitopes refers to a typical length required for MHC class I or MHC class II epitopes. MHC class I epitopes generally have about 5 to 12 residues. Optimum MHC class I epitopes for inducing CD8-positive T cells are 8- to 11-residue peptides. On the other hand, optimum MHC class II epitopes for inducing CD4-positive T cells are considered to be 13- to 18-residue peptides. In the present invention, the length typical of MHC class I epitopes is, for example, 5 to 12 amino acids, preferably 6 to 12 amino acids, more preferably 7 to 12 amino acids, still more preferably 8 to 11 amino acids, and for example, 7, 8, 9, 10, or 11 amino acids. In the present invention, the length typical of MHC class II epitopes is, for example, 15 amino acids or longer, preferably 14 to 25 amino acids or longer, more preferably 13 to 18 amino acids, and for example, 22, 20, 18, 15, or 13 amino acids.

A polypeptide of the present invention can be produced, for example, by the following steps:

(i) dividing an amino acid sequence encoding a desired target antigen protein into amino acid sequences having a length that is at least a length typical of MHC class I epitopes but not a length typical of MHC class II epitopes, wherein the divided amino acid sequences may or may not overlap with one another;

(ii) connecting the divided amino acid sequences in such a way as not to become the same as the amino acid sequence of the antigen protein (i.e. the original amino acid sequence), wherein a spacer may or may not be inserted in each of the connection sites of the divided amino acid sequences; and

(iii) obtaining a polypeptide comprising an amino acid sequence resulting from step (ii).

In addition, a nucleic acid encoding a polypeptide of the present invention can be produced, for example, by the following steps:

(i) dividing an amino acid sequence encoding a desired target antigen protein into amino acid sequences having a length that is at least a length typical of MEW class I epitopes but not a length typical of MHC class II epitopes, wherein the divided amino acid sequences may or may not overlap with one another;

(ii) connecting the divided amino acid sequences in such a way as not to become the same as the amino acid sequence of the antigen protein (i.e. the original amino acid sequence), wherein a spacer may or may not be inserted in each of the connection sites of the divided amino acid sequences; and

(iii) obtaining a nucleic acid encoding a polypeptide comprising an amino acid sequence resulting from step (ii).

Here, the “length that is at least a length typical of MHC class I epitopes but not a length typical of MHC class II epitopes” refers to, for example, a length of 5 to 14 amino acids, preferably 5 to 13 amino acids, more preferably 5 to 12 amino acids, still more preferably 8 to 12 amino acids, still more preferably 8 to 11 amino acids.

A target antigen protein is not particularly limited, and may be any desired protein. An antigen protein may be a natural protein or an artificial protein, but preferably is a natural protein. A full-length protein or a partial protein thereof may be used as an antigen protein. A fusion protein in which multiple proteins are linked together may also be used as an antigen protein. An antigen protein used in the present invention is preferably a protein associated with a disease. Particularly preferred is an antigen protein against which the induction of cellular immunity leads to prevention and/or treatment of a disease. Typical target antigen proteins include a protein of a desired pathogen, pathogenic microorganism, parasite, or such, or a fragment thereof; and a cancer antigen (tumor-specific protein) or cancer stem cell antigen, or a fragment thereof. Examples of tumor antigens include, for example, WT1, survivin, survivin-B2, MAGE-A3, MEGE-A4, tyrosinase, gp100, Melan-A, TRP-2, SNRPD1, CDK4, NY-ESO-1, HER2, MUC-1, CD20, and p53. Cancer stem cell antigens include CD44, CD133, LGRS, and Dclkl. Viral antigens include component proteins of viruses such as hepatitis virus (such as HBV and HCV), human papilloma virus, human immunodeficiency virus, and adult T-cell leukemia virus. Parasite antigens include Plasmodium proteins.

Antigen proteins include, in particular, proteins derived from infectious microorganisms, particularly pathogenic viruses, more specifically CD4-positive T cell-infecting viruses. Such viruses include human immunodeficiency virus (HIV), which causes acquired immunodeficiency syndrome (AIDS), and human T-cell leukemia virus (HTLV-1), which causes adult T-cell leukemia (ATL). A protein of these viruses or a fragment thereof can be suitably used as an antigen protein of the present invention.

The present invention also relates to polypeptides comprising multiple peptides connected together, wherein each of the multiple peptides has an amino acid sequence of 12 residues or less included in the amino acid sequence of a desired antigen protein. The term “12 residues or less” is not particularly limited in its lower limit as long as the amino acid sequence has such a length as to be a potential MHC class I epitope, and refers to, for example, 5 to 12 amino acids, preferably 6 to 12 amino acids, more preferably 7 to 12 amino acids, still more preferably 8 to 12 amino acids, 9 to 12 amino acids, 10 to 12 amino acids, or 10 to 11 amino acids. The term “multiple” may be any plural number as long as the peptides are expected to actually include a peptide serving as an MHC class I epitope, and refers to, for example, 10 or more, preferably 15 or more, more preferably 20 or more, for example, 30 or more, 40 or more, or 50 or more.

Specifically, a polypeptide of the present invention may contain an amino acid sequence in which partial amino acid sequences (also referred to as divided amino acid sequences) excised from the amino acid sequence of a desired antigen protein such that they have at least 5 consecutive amino acids thereof (preferably 6, 7, 8, 9, 10, or 11 consecutive amino acids thereof), but not 15 consecutive amino acids thereof (preferably 14, 13, or 12 consecutive amino acids thereof), are connected in such a way as not to become the same as the original amino acid sequence of the antigen protein. More specifically, a polypeptide of the present invention may contain an amino acid sequence in which partial amino acid sequences excised from the amino acid sequence of a desired antigen protein such that they have at least 8 amino acids thereof (more preferably at least 9, 10, or 11 amino acids thereof), but not 13 amino acids thereof (more preferably 12 amino acids thereof), are connected in such a way as not to become the same as the original amino acid sequence of the antigen protein.

The phrase “the same as the original amino acid sequence of the antigen protein” means that the amino acid sequence finally generated by connection is identical to the amino acid sequence of the antigen protein (the amino acid sequence of the antigen protein before division). In order not to become the same as the original amino acid sequence, for example, the partial sequences are connected in an altered order, for example, in a non-consecutive order, in a random order, or in no particular order. Alternatively, the partial sequences can be connected via an intervening spacer consisting of one or more amino acids so that even an amino acid sequence generated by connecting them sequentially will not be the same as the original amino acid sequence. Alternatively, if partial sequences are divided from the amino acid sequence of an antigen protein such that they have an overlap of several residues, an amino acid sequence generated by connecting them will not be the same as the original amino acid sequence of the antigen protein.

The manner of excising partial amino acid sequences from the amino acid sequence of an antigen protein is not particularly limited. Sequence fragments may be excised such that all fragments have the same length (for example, a common length of 8, 9, 10, 11, or 12 amino acids) or different fragments have different lengths (for example, each fragment is 8, 9, 10, 11, or 12 amino acids long), but typically in the former manner.

For example, cases of dividing the following antigen protein amino acid sequence are exemplified below:

(SEQ ID NO: 1)
YPVQQIGGNYVHLPLSPRTLNAWVKLIEEKKFGAEVVPGFQALSEGCTPY
DINQMLNCVG . . ..

When this sequence is divided into, for example, 11-residue amino acid sequences, for example, it can be divided as follows:

[Case I]
(SEQ ID NO: 2)
YPVQQIGGNYV
(SEQ ID NO: 3)
HLPLSPRTLNA
(SEQ ID NO: 4)
WVKLIEEKKFG
(SEQ ID NO: 5)
AEVVPGFQALS
(SEQ ID NO: 6)
EGCTPYDINQM
.....

The manner of division shown above allows the entire (i.e. 100%) amino acid sequence of the antigen protein to be divided into 11-residue amino acid sequences except for the last remaining portion of less than 11 residues (if such a portion occurs, though). In the present invention, this is called a ratio of coverage of the amino acid sequence of the antigen protein. In the above case, it is almost 100%. A connected amino acid sequence included in a polypeptide of the present application has, for example, 50% or higher, preferably 55% or higher, 60% or higher, 65% or higher, 70% or higher, 75% or higher, 80% or higher, 85% or higher, 90% or higher, 95% or higher, or 100% coverage of the amino acid sequence of an antigen protein.

If these divided amino acid sequences (in the above case, SEQ ID NOs: 2-6) are connected sequentially, the connected sequence will be back to the original amino acid sequence of the antigen protein (or in other words, become the same as the original amino acid sequence). To avoid this, the order of connection can be changed appropriately. For example, SEQ ID NOs: 2, 4, 3, 5, . . . can be connected in this order so that the connected sequence will not become the same as the original amino acid sequence. Such a manner of connection is not particularly limited. The divided sequences may be connected in a non-consecutive but consistent order, or at random. Random connection may probabilistically result in fragments originally next to each other being again connected next to each other. Such connections are acceptable to some extent, but preferably should be avoided as much as possible. For example, in a connected amino acid sequence included in a polypeptide of the present invention, the number of connections that result in fragments originally next to each other being again connected next to each other and thereby yield a connected amino acid sequence portion identical to the corresponding original amino acid sequence of the antigen protein is, for example, 10% or less, preferably 8% or less, more preferably 5% or less, even more preferably 3% or less, and still more preferably 1% or less, of the total number of connections included in the connected amino acid sequence. Obviously, it is most preferable not to include such connections.

For example, it is preferred that a connected amino acid sequence does not substantially contain a partial amino acid sequence of longer than 15 consecutive amino acids (preferably, at least longer than 14, 13, 12, or 11 amino acids) of the original amino acid sequence of the antigen protein. The term “not substantially contain” means that the connected amino acid sequence does not contain such a long consecutive partial amino acid sequence, or that the total number of residues of such a long consecutive partial amino acid sequence is sufficiently smaller than the total number of residues of the connected amino acid sequence. “Sufficiently smaller” means, for example, that the total number of residues of such a long consecutive partial amino acid sequence is preferably 30% or smaller, more preferably 25% or smaller, still more preferably 20% or smaller, even more preferably 15% or smaller, still more preferably 10% or smaller, or even more preferably 5% or smaller, of the total number of residues of the connected amino acid sequence. For example, the connected amino acid sequence does not contain a partial amino acid sequence of longer than 12 consecutive amino acids (preferably longer than 11 amino acids) of the original antigen protein, or alternatively, the total number of residues of such a partial amino acid sequence is 10% or smaller (more preferably 5% or smaller) of the total number of residues of the connected amino acid sequence.

Divided amino acid sequences may be connected via a spacer. A spacer may consist of one or more amino acid residues, preferably one to several amino acid residues, for example, 1, 2, 3, or 4 desired amino acid residues. Amino acid to be used as a spacer is not particularly limited. For example, alanine (A) can be used. For example, the divided amino acid sequences exemplified above (SEQ ID NOs: 2-6) can be connected via a spacer so that even a sequence generated by connecting them sequentially will not be back to the original amino acid sequence of the antigen protein (or in other words, not become the same as the original amino acid sequence). Of course, a spacer may also be appropriately inserted when connecting divided amino acid sequences non-consecutively or randomly.

The manner of dividing the amino acid sequence of an antigen protein is not limited to the one mentioned above. For example, 8- to 12-residue amino acid sequences may be excised from anywhere in the amino acid sequence of an antigen protein. For example, when the antigen protein amino acid sequence shown above (SEQ ID NO: 1) is divided into 11-residue amino acid sequences separated with a gap of 3 residues, it can be divided as follows:

[Case 2]
(SEQ ID NO: 2)
YPVQQIGGNYV
(SEQ ID NO: 7)
LSPRTLNAWVK
(SEQ ID NO: 8)
EKKFGAEVVPG
(SEQ ID NO: 9)
LSEGCTPYDIN
.....

In this case, the ratio of coverage of the amino acid sequence of the antigen protein is 11/14, i.e. 78.6%, provided that the last remaining portion of less than 11 residues is excluded. These divided amino acid sequences can be connected in any desired order. For example, they can be connected in the same order as original, in a non-consecutive order, or in a random order. A spacer may or may not be inserted in connection sites.

Amino acid sequences divided from the amino acid sequence of an antigen protein may overlap with one another. For example, dividing the antigen protein amino acid sequence shown above (SEQ ID NO: 1) into 11-residue amino acid sequences with an overlap of 3 residues with one another will result in the following sequences:

[Case 3]
(SEQ ID NO: 2)
YPVQQIGGNYV
(SEQ ID NO: 10)
NYVHLPLSPRT
(SEQ ID NO: 11)
PRTLNAWVKLI
(SEQ ID NO: 12)
KLIEEKKFGAE
(SEQ ID NO: 13)
GAEVVPGFQAL
(SEQ ID NO: 14)
QALSEGCTPYD
(SEQ ID NO: 15)
PYDINQMLNCV
.....

Making overlaps as shown above is advantageous in that a wider variety of divided sequences having such a length as to be potential MHC class I epitopes can be incorporated into a connected amino acid sequence. For example, in the case of dividing the amino acid sequence of an antigen protein into 11-amino acid fragments without gaps or overlaps as in “Case 1” and connecting them to produce a polypeptide, the amino acid sequence may be divided in 11 different frames. Specifically, “Case 1” above shows the case where the antigen protein amino acid sequence is divided into 11-amino acid fragments starting from the 1st amino acid. In addition to this, the amino acid sequence may be divided into 11-amino acid fragments starting from the 2nd amino acid, the 3rd amino acid, . . . , and the 11th amino acid. Therefore, in order to cover all 11-amino acid divided sequences in all frames, 11 connected amino acid sequences are required. However, in the case where amino acid sequences divided such that they have an overlap of 3 residues are connected to produce a connected amino acid sequence as in “Case 3”, only 8 connected amino acid sequences are required to cover all 11-amino acid divided sequences in all frames (see the Examples). Thus, by providing an overlap between divided amino acid sequences, all theoretically possible divided amino acid sequences (that is, potential MHC class I epitope sequences) present in the amino acid sequence of the antigen protein can be covered using fewer connected amino acid sequences.

When an overlap is provided, the length of the overlap is not particularly limited. However, when the connected polypeptide is expressed as a recombinant protein and such, the length of overlapping regions should preferably not be very long in order to avoid unwanted events caused by homologous recombination such as sequence deletion and duplication. The length of an overlap between divisional amino acid sequences is, for example, one to several residues, and specifically, for example, 1 to 6 amino acids, more preferably 1 to 5 amino acids, even more preferably 1 to 4 amino acids, still more preferably 1 to 3 amino acids, and even more preferably 1 to 2 amino acids.

In the cases shown above, the amino acid sequence of the antigen protein is divided into sequences of a fixed number of amino acids (in the above cases, 11 amino acids). However, the number of amino acids does not need to be fixed. For example, in the case below, the antigen protein amino acid sequence of SEQ ID NO: 1 is divided into sequences of 11 amino acids, 8 amino acids, 10 amino acids, 9 amino acids, and 11 amino acids in this order. Each divided amino acid sequence may or may not have a gap, and may or may not have an overlap. The present invention also encompasses embodiments where such divided amino acid sequences are connected.

[Case 4]
(SEQ ID NO: 2)
YPVQQIGGNYV
(SEQ ID NO: 16)
NYVHLPLS
(SEQ ID NO: 17)
SPRTLNAWVK
(SEQ ID NO: 18)
EEKKFGAEV
(SEQ ID NO: 19)
EVVPGFQALSE
.....

Typically, the amino acid sequence of an antigen protein is divided into sequences of any fixed number of amino acids selected from 8 to 12 (for example, if “11 amino acids” is selected, all divided sequences consist of 11 amino acids). In addition, overlaps between divided amino acid sequences also consist of a fixed number of amino acids. (For example, if an overlap of 3 amino acids is selected, all divided amino acid sequences have an overlap of 3 amino acids at both ends. If no overlap is provided, all divided sequences have no overlap.)

The number of divided amino acid sequences to be connected might depend on the length of the antigen protein. The number of divided amino acid sequences to be connected is not particularly limited. However, connecting as many different divided amino acid sequences as possible is expected to increase the likelihood of including an MHC class I epitope sequence specific for the antigen protein and increase the number of such sequences. The number of divided amino acid sequences to be connected is, for example, 10 or greater, preferably 15 or greater, 20 or greater, 25 or greater, 30 or greater, 35 or greater, 40 or greater, 45 or greater, 50 or greater, or 55 or greater. A connected amino acid sequence thus produced preferably has a high ratio of coverage of the amino acid sequence of the antigen protein as described above, and it is preferred not to place a gap when dividing wherever possible. For example, when the amino acid sequence of an antigen protein is divided into 11-amino acid sequences overlapping with one another by 3 residues, the length of the antigen protein required to ensure 10 divided amino acid sequences is 83 amino acids, and the length of the antigen protein required to ensure 20 divided amino acid sequences is 163 amino acids, Accordingly, it is preferred to select a source antigen protein with a length of, for example, 80 amino acids or longer, preferably 85 amino acids or longer, preferably 100 amino acids or longer, more preferably 150 amino acids or longer, 200 amino acids or longer, 250 amino acids or longer, 300 amino acids or longer, or 350 amino acids or longer, more preferably 400 amino acids or longer. In addition, a connected polypeptide (connected amino acid sequence) has a length of, for example, 100 amino acids or longer, preferably 120 amino acids or longer, preferably 150 amino acids or longer, more preferably 200 amino acids or longer, 250 amino acids or longer, 300 amino acids or longer, 350 amino acids or longer, or 400 amino acids or longer, more preferably 500 amino acids or longer.

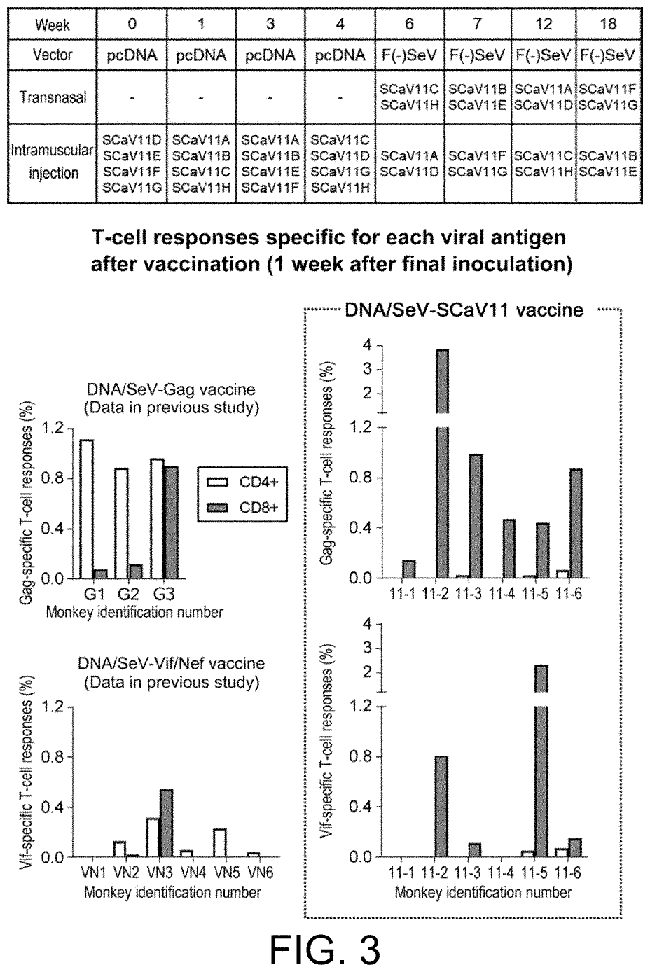

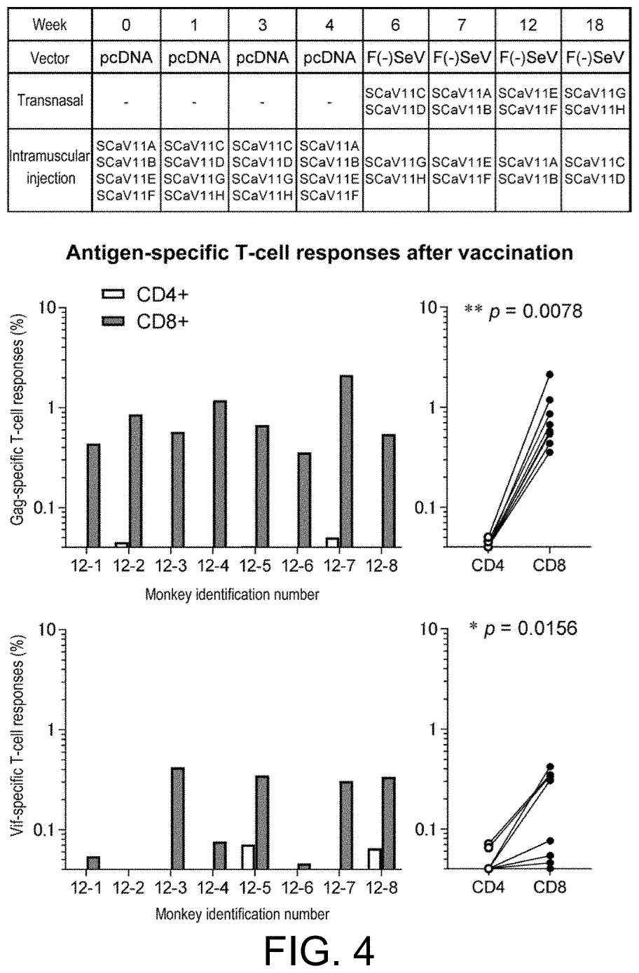

A polypeptide including a connected amino acid sequence is expected to contain a potential MHC class I epitope for a target antigen protein, but not a potential MHC class II epitope for the antigen protein. Inoculation of this polypeptide as an antigen is expected to induce almost no MHC class II-mediated immune responses against the target antigen protein. In fact, as shown in the graphs of FIGS. 3 and 4, whereas the frequency of target antigen-specific CD4-positive T cells is significantly increased when the Gag protein or Vif/Nef protein of HIV is simply inoculated as an antigen, it is hardly increased when a polypeptide of the present invention is inoculated as an antigen. Then it has been found that inoculation of the polypeptide of the present invention as an antigen significantly increases the frequency of target antigen-specific CD8-positive T cells. Therefore, the polypeptide of the present invention is useful for selectively inducing MHC class I-mediated immune responses against a target antigen protein.

MHC class I and MHC class II immune responses against a target antigen protein can be measured by known methods. For example, a polypeptide of the present invention or a nucleic acid or vector encoding the polypeptide is inoculated, and peripheral blood mononuclear cells (PBMCs) are collected from the blood. The obtained cells are stimulated with the antigen, and IFN-γ-producing cells are detected to determine the frequency of target antigen-specific T cells.

When a polypeptide of the present invention is used as an antigen, the frequency of target antigen protein-specific CD8-positive T cells is selectively increased. The term “selectively” means that the increase of the frequency of target antigen protein-specific CD8-positive T cells is significantly higher than the increase of the frequency of target antigen protein-specific CD4-positive T cells. The “increase ratio of the frequency of target antigen protein-specific CD8-positive T cells/increase ratio of the frequency of target antigen protein-specific CD4-positive T cells” (CD8 T frequency increase ratio/CD4 T frequency increase ratio) resulting from a polypeptide of the present invention may be, for example, 1.1 or higher, preferably 1.2 or higher, 1.3 or higher, 1.5 or higher, 2 or higher, 3 or higher, 5 or higher, 10 or higher, 15 or higher, 20 or higher, or 30 or higher. Moreover, the value of “CD8 T frequency increase ratio/CD4 T frequency increase ratio” resulting from the polypeptide of the present invention may be, for example, 1.1 or higher, preferably 1.2 or higher, 1.3 or higher, 1.5 or higher, 2 or higher, 3 or higher, 5 or higher, 10 or higher, 15 or higher, 20 or higher, or 30 or higher, as compared to when the original target antigen protein is used as an antigen.

The measurement of cell frequency mentioned above can be performed at an appropriate time on or after 5 days of inoculation, for example, 1 week, 2 weeks, 3 weeks, or 4 weeks after inoculation. Even when inoculation is carried out multiple times, measurement can be performed at an appropriate time. For example, blood can be collected and measured one week after final inoculation.

When a polypeptide of the present invention is inoculated, the value of “frequency of target antigen protein-specific CD8-positive T cells/frequency of target antigen protein-specific CD4-positive T cells” (CD8 T frequency/CD4 T frequency) may be, for example, 1.1 or higher, preferably 1.2 or higher, 1.3 or higher, 1.5 or higher, 2 or higher, 3 or higher, 5 or higher, 10 or higher, 15 or higher, 20 or higher, or 30 or higher, at any time after 5 days of inoculation. Moreover, the value of “CD8 T frequency/CD4 T frequency” resulting from a polypeptide of the present invention may be, for example, 1.1 or higher, preferably 1.2 or higher, 1.3 or higher, 1.5 or higher, 2 or higher, 3 or higher, 5 or higher, 10 or higher, 15 or higher, 20 or higher, or 30 or higher, as compared to when the original target antigen protein is used as an antigen.

A polypeptide including a connected amino acid sequence may include other amino acid sequences as appropriate. For example, a methionine (M) can be added to the beginning of the polypeptide, and a spacer amino acid may be included between the methionine and the connected amino acid sequence. When an alanine (A) is used as a spacer amino acid, the beginning of the polypeptide (N-terminus) may be MA (Met-Ala). To the C-terminus of the polypeptide, a tag, spacer, and such may be added as appropriate. For example, for experimental use, any desired sequence such as H-2K^{d} RT2 epitope (VYYDPSKDLI/SEQ ID NO: 20) can be added to the C-terminus. Such sequences may be added via a spacer amino acid, and a further spacer amino acid (e.g. Ala) may be added to the C-terminus.

A polypeptide of the present invention can include a connected amino acid sequence prepared from amino acid sequences of more than one antigen protein. For example, connected amino acid sequences prepared separately from two proteins of a certain pathogen can be connected to make one polypeptide. For example, in the Examples, a connected amino acid sequence prepared from the amino acid sequence of the Gag protein of HIV was joined to a connected amino acid sequence prepared from the amino acid sequence of the Vif protein to produce one polypeptide. In such a manner, a polypeptide of the present invention can include a connected amino acid sequence prepared from more than one antigen protein.

When a polypeptide of the present invention is inoculated as an antigen, more than one polypeptide of the present invention can be used in combination. Here, the phrase “used in combination” is not limited to simultaneous use, and may be use of a series of peptides in a serial or sequential manner. As described above, when the amino acid sequence of an antigen protein is divided into, for example, 11-amino acid sequences, there are 11 dividing frames. Therefore, in order to cover all potential MHC class I epitope (CD8-positive T cell epitope) sequences that may exist in the amino acid sequence of the target antigen protein, for example, 11 connected amino acid sequences are required if the divided amino acid sequences have no overlap, or 8 connected amino acid sequences are required if the divided sequences have an overlap of 3 residues. These connected amino acid sequences can be expressed as polypeptides from a single expression vector, or expressed as a single polypeptide in which the connected amino acid sequences are connected together. However, the connected amino acid sequences, which share many common nucleic acid sequences of about several tens of bases, have a risk of homologous recombination. To avoid that, the recombinant expression of the connected amino acid sequences prepared in different frames is preferably performed by expressing them as separate polypeptides from separate vectors. An appropriate combination of polypeptides including these connected amino acid sequences prepared in different frames or nucleic acids or vectors encoding them can cover a wide range of theoretically possible potential MHC class I epitope sequences, and can be inoculated to efficiently induce target-specific CD8-positive T cells.

For example, in “Case 3” above, a combination of 8 polypeptides including connected amino acid sequences prepared in different frames can cover all (i.e. 100%) theoretically possible potential MHC class I epitope sequences (a set of 11-amino acid sequences that may be chosen from the amino acid sequence of the antigen protein). In the present invention, this is called a ratio of coverage of the divided sequences of the antigen protein. This coverage ratio corresponds to a ratio of coverage of the potential MEW class I epitopes present in the antigen protein. In “Case 1” above, the ratio of coverage of the divided sequences in one connected amino acid sequence (when divided into 11-residue sequences; this is referred to as a ratio of coverage of the divisional sequences at a window width of 11 amino acids) is 1/11, i.e. 9.1%. When n connected amino acid sequences in different frames are combined, the coverage ratio is (1/11)*n %. In “Case 3” above, the ratio of coverage of the divided sequences in one connected amino acid sequence (when divided into 11-residue sequences) is 1/8, i.e. 12.5%. When n connected amino acid sequences in different frames are combined, the coverage ratio is (1/8)*n %. When multiple polypeptides of the present invention are combined, the combination is such that the ratio of coverage of the divided sequences of the antigen protein is, for example, 20% or higher, preferably 25% or higher, more preferably 30% or higher, even more preferably 35% or higher, still more preferably 40% or higher, even more preferably 45% or higher, still more preferably 50% or higher, 60% or higher, 70% or higher, 80% or higher, 90% or higher, 95% or higher, or 100%. This coverage ratio is calculated in accordance with the length of the divided amino acid sequences. The amino acid length (i.e. window width) is, for example, 8 amino acids, preferably 9 amino acids, more preferably 10 amino acids, still more preferably 11 amino acids. For example, to achieve 60% or higher coverage of the divided sequences of the antigen protein in “Case 3” above (divided into 11-amino acid sequences with an overlap of 3 residues), 6 connected amino acid sequences in different frames are combined.

For example, polypeptides including connected amino acid sequences of the present invention may be a combination of at least 2, preferably 2, more preferably 4, even more preferably 5, 6, 7, 8, 9, 10, or 11 polypeptides in different dividing frames. These polypeptides may be prepared as separate vaccine compositions or mixed in a single composition.

The present invention also relates to nucleic acids that encode a polypeptide including a connected amino acid sequence of the present invention. Such a nucleic acid is not particularly limited, and may be DNA or RNA. Meanwhile, negative-strand RNA viral vectors, which are described later, are viruses having an antisense single-stranded RNA genome, which encodes proteins in the antisense orientation. Thus, the nucleic acids of the present invention include not only those encoding a polypeptide in the sense strand but also those encoding a polypeptide in the antisense strand. In addition, the nucleic acids may be single-stranded or double-stranded. In designing the nucleotide sequence of a nucleic acid, the codons may be appropriately optimized according to the host for expressing the polypeptide.

A nucleic acid of the present invention may encode other polypeptides as long as it encodes the polypeptide of the present invention. It may also contain other sequences such as a replication origin, promoter, enhancer, terminator, and spacer.

The present invention also provides vectors containing such a nucleic acid. A vector of the present invention is not particularly limited as long as it carries a nucleic acid of the present invention. For example, the vector may be a plasmid vector, phage vector, cosmid, viral vector, artificial chromosome, or such. In particular, the vectors of the present invention include expression vectors. By using an expression vector that can be administered to animals in vivo, a polypeptide of the present invention can be expressed in the animal body to function as a vaccine.

Such vectors include non-viral vectors and viral vectors, including, for example, plasmid vectors, adenoviral vectors, retroviral vectors (including lentiviral vectors), adeno-associated viral vectors, vaccinia virus vectors, cytomegalovirus vectors, and pox virus vectors (Wilson N A. et al., J Virol. 80: 5875-5885, 2006; Hansen S G. et al., Nature. 473: 523-527, 2011; Barouch D H. et al., Nature. 482: 89-93, 2012), but are not limited thereto.

In particular, the vectors of the present invention include negative-strand RNA viral vectors. The present inventors' study using a Sendai virus (SeV) vector, which is one of the negative-strand RNA viral vectors, in a simian AIDS model has shown that a SeV vector vaccine expressing a single CD8-positive T cell SIV epitope did not induce SIV-specific CD4-positive T cells but induced effective SIV epitope-specific CD8-positive T cells (Tsukamoto T. et al., J Virol. 83: 9339-9346, 2009; Ishii H. et al., J Virol. 86: 738-745, 2012). Therefore, the use of a negative-strand RNA viral vector to express a polypeptide of the present invention is expected to more highly induce effective antigen-specific CD8-positive T cells selectively while suppressing the induction of antigen-specific CD4-positive T cells as much as possible.

As described above, negative-strand RNA viral vectors are chromosomally non-integrating viral vectors and expressed within the cytosol. Therefore, they have no risk of integrating genes they carry into host chromosomes (nuclear chromosomes). They are therefore highly safe, and also easily removed from infected cells. Negative-strand RNA viral vectors including Sendai virus (SeV) vectors (Matano T. et al., J Exp Med. 199: 1709-1718, 2004; Nyombayire J. et al., J Infect Dis. 215: 95-104, 2017) are useful as vectors capable of inducing effective CD8-positive T cells.

In the present invention, the negative-strand RNA viral vectors include infectious viral particles, and also include viral cores, complexes composed of a viral genome and viral proteins, or complexes composed of a non-infectious viral particle and such, that are capable of expressing a gene they carry when introduced into cells. For example, the ribonucleoprotein (viral core) of a negative-strand RNA virus, which consists of a viral genome and negative-strand RNA virus proteins binding to it (e.g. NP, P, and L proteins), can express a transgene intracellularly when introduced into cells (WO00/70055). The introduction into cells may be performed using a transfection agent and such, as appropriate. Such ribonucleoproteins (RNPs) are also included in the negative-strand RNA viral vectors in the present invention. Preferably, a negative-strand RNA viral vector in the present invention is a particle in which the aforementioned RNP is enclosed by a biological membrane derived from the cell membrane.

Negative-strand RNA viral vectors used in the present invention particularly include paramyxovirus vectors. The paramyxovirus refers to a virus belonging to the family Paramyxoviridae or a derivative thereof. Paramyxoviridae includes the subfamilies Paramyxovirinae (including the genera Respirovirus (also called Paramyxovirus), Rubulavirus, and Morbillivirus) and Pneumovirinae (including the genera Pneumovirus and Metapneumovirus). The viruses belonging to the family Paramyxoviridae specifically include Sendai virus, Newcastle disease virus, mumps virus, measles virus, RS virus (respiratory syncytial virus), (rinderpest virus), distemper virus, simian parainfluenza virus (SV5), human parainfluenza virus types 1, 2, and 3. More specifically, those viruses include, for example, Sendai virus (SeV), human parainfluenza virus-1 (HPIV-1), human parainfluenza virus-3 (HPIV-3), phocine distemper virus (PDV), canine distemper virus (CDV), dolphin molbillivirus (DMV), peste-des-petits-ruminants virus (PDPR), measles virus (MeV), rinderpest virus (RPV), Hendra virus (Hendra), Nipah virus (Nipah), human parainfluenza virus-2 (HPIV-2), simian parainfluenza virus 5 (SV5), human parainfluenza virus-4a (HPIV-4a), human parainfluenza virus-4b (HPIV-4b), mumps virus (Mumps), and Newcastle disease virus (NDV). Rhabdovirus includes vesicular stomatitis virus and rabies virus, which belong to the family Rhabdoviridae.

As described above, the genomic RNA of negative-strand RNA viruses is a negative strand. Their protein amino acid sequences are encoded in an antigenome having a sequence complementary to the genomic RNA. In the present invention, both genome and antigenome may be referred to as genome for the sake of convenience.

In the present invention, a viral vector is preferably a virus belonging to the subfamily Paramyxovirinae (including the genera Respirovirus, Rubulavirus, and Morbillivirus) or a derivative thereof, more preferably a virus belonging to the genus Respirovirus (also called Paramyxovirus) or a derivative thereof. The derivatives include a virus whose viral genes have been altered, and a virus which have been chemically modified, without impairing the gene transfer ability of the virus. Viruses of the genus Respirovirus to which the present invention can be applied include, for example, human parainfluenza virus type 1 (HPIV-1), human parainfluenza virus type 3 (HPIV-3), bovine parainfluenza virus type 3 (BPIV-3), Sendai virus (also called murine parainfluenza virus type 1), measles virus, simian parainfluenza virus (SV5), and simian parainfluenza virus type 10 (SPIV-10). In the present invention, the most preferred paramyxovirus is Sendai virus.

A paramyxovirus in general contains within its envelope a complex consisting of RNA and proteins (ribonucleoprotein; RNP). The RNA contained in the RNP is (−)strand (negative-strand), single-stranded RNA, which is a genome of negative-strand RNA virus. This single-stranded RNA binds NP protein, P protein, and L protein to form the RNP. The RNA contained in this RNP serves as a template for transcription and replication of the viral genome (Lamb, R. A., and D. Kolakofsky, 1996, Paramyxoviridae: The viruses and their replication. pp. 1177-1204. In Fields Virology, 3rd edn. Fields, B. N., D. M. Knipe, and P. M. Howley et al., (ed.), Raven Press, New York, N. Y).

A viral vector may be derived from a virus of a natural strain, wild-type strain, mutant strain, laboratory-passaged strain, and artificially-established strain, and the like. For Sendai virus, examples include Z strain but are not limited thereto (Medical Journal of Osaka University Vol. 6, No. 1, March 1955 p 1-15). For example, a wild-type virus with a mutation or deficiency in any of its genes may be used. For example, a virus that is deficient in at least one of the genes encoding its envelope protein or coat protein or contains a mutation suppressing the expression thereof such as a stop codon mutation can suitably be used. Such viruses that do not express the envelope protein are, for example, capable of replicating the genome but not capable of forming infectious viral particles in cells they have infected. Such propagation-deficient viruses are particularly suitable as highly safe vectors. For example, a virus that does not encode in its genome one or both of the envelope protein (spike protein) genes F and HN can be used (WO00/70055 and WO00/70070; Li, H.-O. et al., J. Virol. 74(14) 6564-6569 (2000)). A virus can replicate its genome in cells it has infected as long as the genomic RNA encodes at least proteins necessary for genome replication (e.g. N, P, and L proteins). To produce envelope protein-deficient, infectious viral particles, for example, the deficient gene product or a protein that can complement it is exogenously supplied in virus-producing cells (WO00/70055 and WO00/70070; Li, H.-O. et al., J. Virol. 74(14) 6564-6569 (2000)). On the other hand, non-infectious viral particles can be recovered by not complementing the deficient viral protein at all (WO00/70070).

In producing a virus of the present invention, it is also preferred to use a virus carrying a mutant viral protein gene. For example, there are a large number of known mutations including attenuating mutations and temperature-sensitive mutations for viral structural proteins (NP, M) and RNA synthase (P, L). Paramyxovirus vectors and such containing these mutant protein genes can suitably be used according to the purpose in the present invention.

Viral vectors containing a nucleic acid encoding a polypeptide of the present invention can be constructed using known methods (WO97/16539; WO97/16538; WO00/70070; WO01/18223; WO2005/071092; Hasan, M K et al., J Gen Virol 78:2813-2820, 1997; Kato A et al., EMBO J 16: 578-587, 1997; Yu D et al., Genes Cells 2: 457-466, 1997; Kato A et al., Genes Cells 1; 569-579, 1996; Tokusumi T et al., Virus Res 86: 33-38, 2002; Li H O et al., J Virol 74: 6564-6569, 2000).

The present invention also provides a composition comprising a polypeptide of the present invention or a nucleic acid or a vector encoding the polypeptide. The composition may contain a desired carrier and/or vehicle. The carriers and vehicles include desired pharmaceutically acceptable carriers and vehicles including, for example, sterile water, physiological saline, phosphate buffered saline (PBS), buffers, and culture fluids. In addition, glycols, glycerol, oils such as olive oil, and organic esters may also be added. Additives such as suspending liquids, emulsifiers, diluents, and excipients may be mixed as appropriate for formulation. Methods of formulation and additives that can be used are well-known in the field of pharmaceutical formulation. The forms of formulation are not particularly limited, and include, for example, injections, inhalants, and capsules. Furthermore, the present invention also relates to vaccine formulations comprising a polypeptide of the present invention or a nucleic acid or a vector encoding the polypeptide. The compositions or vaccine formulations of the present invention are useful for selectively inducing CD8-positive T cells specific for a target antigen protein while suppressing the induction of CD4-positive T cells specific for the antigen protein. The compositions or vaccine formulations of the present invention can be prepared, for example, as a composition containing a polypeptide of the present invention or a nucleic acid or vector encoding the polypeptide, and a desired carrier. The compositions or vaccine formulations of the present invention can be prepared as liposomes such as HVJ liposomes. In addition, the compositions or vaccine formulations of the present invention may further contain a desired adjuvant. Adjuvants include, for example, oil adjuvants and aluminum adjuvants, and more specifically include alum (aluminum salt), MF59 (oil emulsion), and Montanides (such as Montanide ISA 51VG; oil emulsion).

A composition or vaccine formulation of the present invention can contain one or more polypeptides of the present invention. As described above, a combination of polypeptides of the present invention prepared from the amino acid sequence of one antigen protein in different dividing frames can effectively induce target antigen-specific CD8-positive T cells. For example, a composition or vaccine formulation of the present invention may be for combined use of 2 or more, 3 or more, 4 or more, 5 or more, 6 or more, 7 or more, 8 or more, 9 or more, 10 or more, or 11 or more polypeptides of the present invention that target one antigen protein but are prepared in different dividing frames, or alternatively the composition or vaccine formulation may contain those polypeptides. When multiple polypeptides of the present invention prepared in different dividing frames are combined, the combination is such that the ratio of coverage of the divided sequences of the antigen protein is, for example, 20% or higher, preferably 25% or higher, more preferably 30% or higher, even more preferably 35% or higher, still more preferably 40% or higher, even more preferably 45% or higher, still more preferably 50% or higher, 60% or higher, 70% or higher, 80% or higher, 90% or higher, 95% or higher, or 100%. Furthermore, a composition or vaccine formulation of the present invention may be for use in combination with polypeptides of the present invention targeting a different antigen protein, or alternatively the composition or vaccine formulation may further contain those polypeptide.

When a vaccine formulation of the present invention is used, the mode of inoculation thereof is not particularly limited. For example, the vaccine formulation can be used in single or multiple inoculations. In multiple inoculations, the vaccine of the present invention may be inoculated multiple times, or alternatively may be used in combination with other types of vaccine. For example, in performing multiple injections, it may be beneficial to change the polypeptide or combination of peptides to inoculate, rather than repeating the inoculation of the same polypeptide or combination. It may also be beneficial to change the administration route or use more than one administration route for inoculation. Specifically, in the case where all theoretically possible divided amino acid sequences (i.e. potential MHC class I epitope sequences) can be covered by 8 polypeptides, for example, 4 polypeptides can be inoculated at a time, and the combination of 4 polypeptides can be changed in each inoculation (see Example 4 and FIG. 3). It is also possible to perform primary inoculation or the first few inoculations using a non-viral vector (e.g. polypeptide or DNA vector) and subsequent inoculations using a viral vector encoding the polypeptide of the present invention. The viral vector to be used is not particularly limited. For example, paramyxovirus vectors such as Sendai virus vectors may be suitably used.

A polypeptide, nucleic acid, and vector of the present invention can be used in combination with another antigen or a nucleic acid or vector encoding that antigen. For example, primary inoculation or the first few inoculations can be performed using a target antigen that has not been divided like the polypeptide of the present invention, and then the polypeptide, nucleic acid or vector of the present invention can be inoculated in booster inoculations (see Examples 4 and 5). Primary inoculation can be performed, for example, using a DNA vector encoding a target antigen that has not been divided like the polypeptide of the present invention, but is not limited thereto.

When a composition or vaccine formulation of the present invention is inoculated into an animal, its dose can be appropriately determined according to the disease, patient's weight, age, sex, and symptoms, purpose of administration, form of administered composition, administration method, and the like. The route of administration can be appropriately selected, and includes, for example, transnasal administration, intraperitoneal administration, intramuscular administration, and local administration to lesions of infection, tumor, and such, but is not limited thereto. The dose may be appropriately adjusted according to the subject animal, site of administration, and number of administrations. For example, the dose may be from 1 ng/kg to 1000 mg/kg, from 5 ng/kg to 800 mg/kg, from 10 ng/kg to 500 mg/kg, from 0.1 mg/kg to 400 mg/kg, from 0.2 mg/kg to 300 mg/kg, from 0.5 mg/kg to 200 mg/kg, or from 1 mg/kg to 100 mg/kg, but is not limited thereto. In the case of an viral vector, for example, the dose may be from 1×10⁴ to 1×10¹⁵ CIU/kg, from 1×10⁵ to 1×10¹⁴ CIU/kg, from 1×10⁶ to 1×10¹³ CIU/kg, from 1×10⁷ to 1×10¹² CIU/kg, from 1×10⁸ to 5×10¹¹ CIU/kg, from 1×10⁹ to 5×10¹¹ CIU/kg, or from 1×10¹⁰ to 1×10¹¹ CIU/kg; or may be from 1×10⁶ to 1×10¹⁷ particles/kg, from 1×10⁷ to 1×10¹⁶ particles/kg, from 1×10⁸ to 1×10¹⁵ particles/kg, from 1×10⁹ to 1×10¹⁴ particles/kg, from 1×10¹⁰ to 1×10¹³ particles/kg, from 1×10¹¹ to 5×10¹² particles/kg, or from 5×10¹¹ to 5×10¹² particles/kg, but is not limited thereto.

Subjects to which a composition or vaccine formulation of the present invention is administered are not particularly limited, but preferably are mammals (including human and non-human mammals). Specifically, the subjects include human, non-human primates such as monkeys, rodents such as mice and rats, rabbits, goats, sheep, pigs, cows, dogs, cats, and all other mammals.

A composition or vaccine formulation of the present invention can be used in combination with other pharmaceuticals. For example, when a polypeptide of the present invention designed against a tumor antigen is used, the composition or vaccine formulation may be used in combination with other anticancer agents. When a polypeptide of the present invention designed against an infectious disease is used, the composition or vaccine formulation may be used in combination with other drugs for that infectious disease.

### EXAMPLES

Herein below, the present invention will be specifically described with reference to Examples, but it is not to be construed as being limited thereto. All cited documents and other references herein are incorporated as part of this specification.

### [Example 1] Construction of Plasmid Carrying SCaV11 Antigen Gene

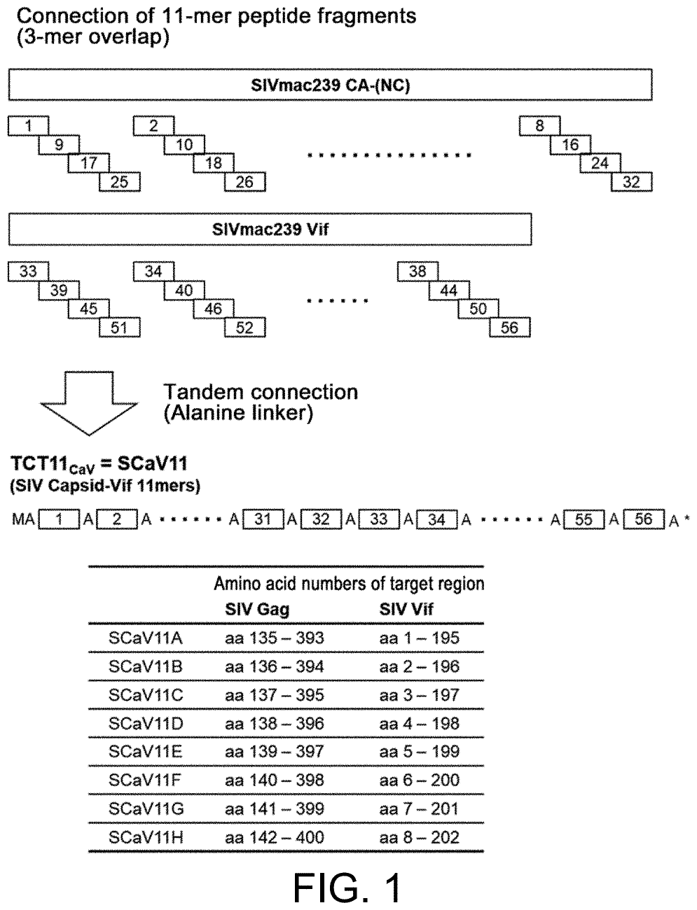

The Gag CA and Vif proteins of SIVmac239 (GenBank Accession No. M33262) were used as target antigens to design a TCT11 antigen (referred to as SCaV11) for evaluation in the SIVmac239-infected monkey AIDS model. The amino acid sequences of the Gag CA protein (amino acid sequence Accession: AAA47632.1 (SEQ ID NO: 21)) and Vif protein (amino acid sequence Accession: AAA47634.1 (SEQ ID NO: 22)) of SIVmac239 were fragmented into 11-mer peptides with an overlap of 3 amino acids with one another. These peptides were rearranged in a different order and connected in tandem using alanine as a spacer (SCaV11)(FIG. 1). The 3-amino acid overlap was for preventing homologous recombination. In a similar manner, a total of 8 tandemly-connected antigens (SCaV11A to pSCaV11H) were designed, for each of which the starting amino acid position of the peptides in the target antigen region was shifted by one amino acid (SEQ ID NOs: 23 to 30, in order). Next, the nucleotide sequences for these antigens were codon-optimized for human, and mutations for preventing homologous recombination were introduced into the sequences. The entire genes were then chemically synthesized (Eurofins Genomics), and inserted into plasmids. These plasmids were named pSCaV11A to pSCaV11H (SEQ ID NOs: 31-38, in order)

### [Example 2] Construction of Sendai Virus (SeV) Vector Carrying SCaV11 Antigen Gene

### (1) Construction of Plasmids for Producing F-Deficient Sendai Viruses Carrying SCaV11 Antigen Genes

PCR was performed on the plasmid carrying the SCaV11A antigen gene as a template, using primers Not1_SCaV11A_N (5′-ATATgcggccgcgacgccaccATGGCCTACCCTGTGCAGCAG-3′ (SEQ ID NO: 39)) and SCaV11A_EIS_Not1_C (5′-ATATGCGGCCGCgatgaactttcaccctaagtttttcttactacggTCAGGCTTTGCCTCCCCTCTGC-3′ (SEQ ID NO: 40)), and KOD-Plus-Ver.2 kit, under the following conditions: 94° C. for 2 min; 30 cycles of 98° C. for 10 sec, 55° C. for 30 sec, and 68° C. for 2.5 min; react at 68° C. for 7 min; and keep at 4° C. The amplified SCaV11A fragment was separated by agarose gel electrophoresis, and then purified using NucleoSpin Gel and PCR Clean-up kit (Takara Bio). In the above primer sequences, the upper-case letters represent a sequence of the SCaV11 antigen gene, and the lower-case letters represent a sequence of the SeV vector (the same applies hereinafter).

Next, the above SCaV11A fragment treated with NotI (having a NotI site on both ends) was ligated into the NotI cleavage site of plasmid pSeV18+/ΔF (WO00/070070), which encodes an F gene-deficient Sendai virus vector. The plasmid was used for transformation of E. coli followed by cloning. Sequencing was performed to select a clone with the correct nucleotide sequence, and thereby plasmid pSeV18+SCaV11A/ΔF was obtained.

Similarly, PCR was performed on the plasmid carrying the SCaV11B antigen as a template using primers Not1_SCaV11B_N (5′-ATATgcggccgcgacgccaccATGGCCCCTGTGCAGCAGATCG-3′ (SEQ ID NO: 41)) and SCaV11B_EIS_Not1_C (5′-ATATGCGGCCGCgatgaactttcaccctaagtttttcttactacggTCAGGCGGGCTTCCCTCCCCTC-3′ (SEQ ID NO: 42)), and the amplified fragment was inserted into the NotI site of plasmid pSeV18+/ΔF to obtain plasmid pSeV18+SCaV11B/ΔF.

Similarly, PCR was performed on the plasmid carrying the SCaV11C antigen as a template using primers Not1_SCaV11C_N (5′-ATATgcggccgcgacgccaccATGGCCGTGCAGCAGATCGGAG-3′ (SEQ ID NO: 43)) and SCaV11C_EIS_Not1_C (5′-ATATGCGGCCGCgatgaactttcaccctaagtttttcttactacggTCAAGCAGGAGGTTTCCCTCCCC-3′ (SEQ ID NO: 44)), and the amplified fragment was inserted into the NotI site of plasmid pSeV18+/ΔF to obtain plasmid pSeV18+SCaV11C/ΔF.

Similarly, PCR was performed on the plasmid carrying the SCaV11D antigen as a template using primers Not1_SCaV11D_N (5′-ATATgcggccgcgacgccaccATGGCCCAGCAGATCGGAGGC-3′ (SEQ ID NO: 45)) and SCaV11D EIS_Not1_C (5′-ATATGCGGCCGCgatgaactttcaccctaagtttttcttactacggTCAGGCTGTTGGGGGTTTCCCTC-3′ (SEQ ID NO: 46)), and the amplified fragment was inserted into the NotI site of plasmid pSeV18+/ΔF to obtain plasmid pSeV18+SCaV11D/ΔF.

Similarly, PCR was performed on the plasmid carrying the SCaV11E antigen as a template using primers Not1_SCaV11E_N (5′-ATATgcggccgcgacgccaccATGGCCCAGATCGGAGGCAATTATG-3′ (SEQ ID NO: 47)) and SCaV11E_EIS_Not1_C (5′-ATATGCGGCCGCgatgaactttcaccctaagtttttcttactacggTCAGGCCTTGGTAGGGGGTTTCC-3′ (SEQ ID NO: 48)), and the amplified fragment was inserted into the NotI site of plasmid pSeV18+/ΔF to obtain plasmid pSeV18+SCaV11E/ΔF.

Similarly, PCR was performed on the plasmid carrying the SCaV11F antigen as a template using primers Not1_SCaV11F_N (5′-ATATgcggccgcgacgccaccATGGCCATCGGAGGCAATTATG-3′ (SEQ ID NO: 49)) and SCaV11F EIS_Not1_C (5′-ATATGCGGCCGCgatgaactttcaccctaagtttttcttactacggTCAGGCGCCTTTTGTAGGGGG-3′ (SEQ ID NO: 50), and the amplified fragment was inserted into the NotI site of plasmid pSeV18+/ΔF to obtain plasmid pSeV18+SCaV11F/ΔF.

Similarly, PCR was performed on the plasmid carrying the SCaV11G antigen as a template using primers Not1_SCaV11G_N (5′-ATATgcggccgcgacgccaccATGGCCGGAGGCAATTATGTG-3′ (SEQ ID NO: 51)) and SCaV11G EIS_Not1_C (5′-ATATGCGGCCGCgatgaactttcaccctaagtttttcttactacggTCAGGCGGCGCCCTTTGTAGGGG-3′ (SEQ ID NO: 52)), and the amplified fragment was inserted into the NotI site of plasmid pSeV18+/ΔF to obtain plasmid pSeV18+SCaV11G/ΔF.

Similarly, PCR was performed on the plasmid carrying the SCaV11H antigen as a template using primers Not1_SCaV11H_N (5′-ATATgcggccgcgacgccaccATGGCCGGAGGCAATTATGTG-3′ (SEQ ID NO: 53)) and SCaV11H_EIS_Not1_C (5′-ATATGCGGCCGCgatgaactttcaccctaagtttttcttactacggTCAGGCGGCGCCCTTTGTAGGGG-3′ (SEQ ID NO: 54)), and the amplified fragment was inserted into the NotI site of plasmid pSeV18+/ΔF to obtain plasmid pSeV18+SCaV11H/ΔF.

### (2) Production (Reconstitution) and Amplification of F-Deficient Sendai Virus Vectors Carrying SCaV11 Antigen Genes

From the plasmids produced as described above for producing SCaV11 antigen gene-carrying F-deficient Sendai viruses, namely, pSeV18+SCaV11A/ΔF to pSeV18+SCaV11H/ΔF, the SCaV11 antigen gene-carrying F-deficient Sendai viruses were produced (reconstituted) and amplified by a known method (for example, WO2005/071092). The resulting viruses were named SeV18+SCaV11A/ΔF, SeV18+SCaV11B/ΔF, SeV18+SCaV11C/ΔF, SeV18+SCaV11D/ΔF, SeV18+SCaV11E/ΔF, SeV18+SCaV11F/ΔF, SeV18+SCaV11G/ΔF, and SeV18+SCaV11H/ΔF, respectively.

### [Example 3] Inoculation Test of SIV CA-Vif TCT11 Antigen-Expressing Vaccines into SIV Controllers (SIV Replication-Controlled Monkeys)

Rhesus monkeys that had controlled SIV replication (SIV controllers) after inoculation of a single epitope (Gag CA)-expressing vaccine followed by transvenous inoculation of SIVmac239 were inoculated with the instant SCaV11-expressing Sendai virus (SeV) vectors during their chronic phase, and examined for induced T-cell responses specific for SIV Gag and Vif antigens.

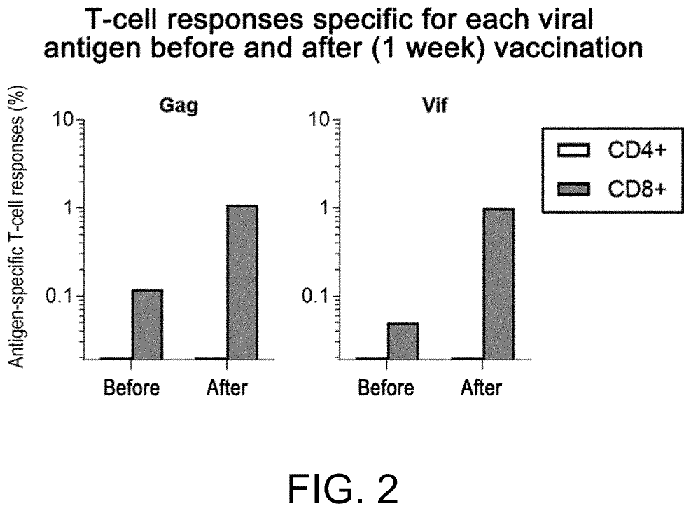

The F-deficient Sendai virus vectors expressing SCaV11A, SCaV11B, SCaV11F, and SCaV11H (SeV18+SCaV11A/ΔF, SeV18+SCaV11B/ΔF, SeV18+SCaV11F/ΔF, and SeV18+SCaV11H/ΔF; 6×10⁹ CIU each) were inoculated transnasally and intramuscularly. Peripheral blood mononuclear cells (PBMCs) were isolated from the blood before vaccination and after one week of vaccination, and analyzed for T-cell responses specific for SIV Gag and Vif antigens. Specifically, the cells were challenged with a pool of overlapping peptides spanning the Gag and Vif regions of SIVmac239, and the frequency of SIV Gag/Vif antigen-specific T cells was determined by detection of IFN-γ-producing cells by intracellular cytokine staining using a flow cytometer. As a result, the frequency of Gag/Vif antigen-specific CD8-positive T cells after vaccination was increased 10-fold or more as compared to that before vaccination; however, the frequency of Gag/Vif antigen-specific CD4-positive T cells was not changed by vaccination (FIG. 2). This result demonstrated that the SCaV11 antigen-expressing SeV vector vaccines induced SIV Gag/Vif antigen-specific CD8-positive T-cell responses in a selective manner.

### [Example 4] Inoculation Test of SCaV11 Vaccines in Uninfected Monkeys

Six uninfected rhesus monkeys were inoculated with the SCaV11 antigen-expressing vaccines and examined for induced SIV antigen-specific T-cell responses.

The 6 monkeys were intramuscularly injected with the plasmid DNA vaccines expressing antigens SCaV11A to SCaV11H (8 antigens) (pcDNA-SCaV11A to pcDNA-SCaV11H, respectively, 5 mg each) twice for each vaccine. The monkeys were then inoculated with the F-deficient SeV vector vaccines expressing antigens SCaV11A to SCaV11H (8 antigens) (SeV18+SCaV11A/ΔF to SeV18+SCaV11H/ΔF, 6×10⁹ CIU each) transnasally and intramuscularly once for each vaccine (FIG. 3). PBMCs were isolated from the blood after one week of the final vaccination, and analyzed for SIV Gag/Vif antigen-specific T-cell responses by the same method as in Example 3.

It had been previously reported that vaccination with DNA/SeV vectors expressing SIV Gag antigen or Vif/Nef antigen effectively induced not only Gag/Vif antigen-specific CD8-positive T-cell responses but also Gag/Vif-specific CD4-positive T-cell responses (Iwamoto N. et al., J Virol. 88:425-433, 2014). On the other hand, the vaccination with the instant SCaV11 antigen-expressing DNA/SeV vectors, while inducing very efficient Gag/Vif antigen-specific CD8-positive T-cell responses, resulted in undetectable or very low levels of Gag/Vif-specific CD4-positive T-cell responses (FIG. 3). These results demonstrated that the DNA prime/SeV vector vaccines expressing SCaV11 antigens induced almost no SIV Gag/Vif antigen-specific CD4-positive T-cell responses, and selectively induced Gag/Vif antigen-specific CD8-positive T cells efficiently.

### [Example 5] Inoculation Test 2 of SCaV11 Vaccines in Uninfected Monkeys

Eight uninfected rhesus monkeys were inoculated with SCaV11 antigen-expressing vaccines and examined for induced SIV antigen-specific T-cell responses.

The 8 monkeys were intramuscularly injected with the plasmid DNA vaccines expressing antigens SCaV11A to SCaV11H (8 antigens) (pcDNA-SCaV11A to pcDNA-SCaV11H, respectively, 5 mg each) twice for each vaccine. The monkeys were then inoculated with the F-deficient SeV vector vaccines expressing antigens SCaV11A to SCaV11H (8 antigens) (SeV18+SCaV11A/ΔF to SeV18+SCaV11H/ΔF, 1×10⁹ CIU each) transnasally and intramuscularly once for each vaccine (FIG. 4). PBMCs were isolated from the blood after one week of the final vaccination, and analyzed for SIV Gag/Vif antigen-specific T-cell responses by the same method as in Example 3. In addition, in order to assess the immune induction ability in the lymph node, lymph node biopsy was performed 2 weeks after the third SeV vector vaccination, and SIV Gag/Vif antigen-specific T-cell responses were analyzed in the same manner.

The SCaV11 antigen-expressing DNA/SeV vector vaccination induced very efficient Gag/Vif antigen-specific CD8-positive T-cell responses, but resulted in undetectable or very low levels of Gag/Vif-specific CD4-positive T-cell responses (FIG. 4). Comparison between antigen-specific CD4-positive T-cell responses and CD8-positive T-cell responses also showed that the frequency of antigen-specific CD8-positive T cells was significantly higher than the frequency of antigen-specific CD4-positive T cells for both Gag- and Vif-specific T cells (Gag; p=0.0078, Vif; p=0.0156 by Wilcoxon matched-pairs signed rank test). These results confirmed the reproducibility of the selective induction of Gag/Vif-specific CD8-positive T-cell responses by the SCaV11 antigen-expressing vaccines, and verified and confirmed the ability of the target 11-mer connected antigen TCT11 vaccine to induce antigen-specific CD8-positive T-cell responses selectively.

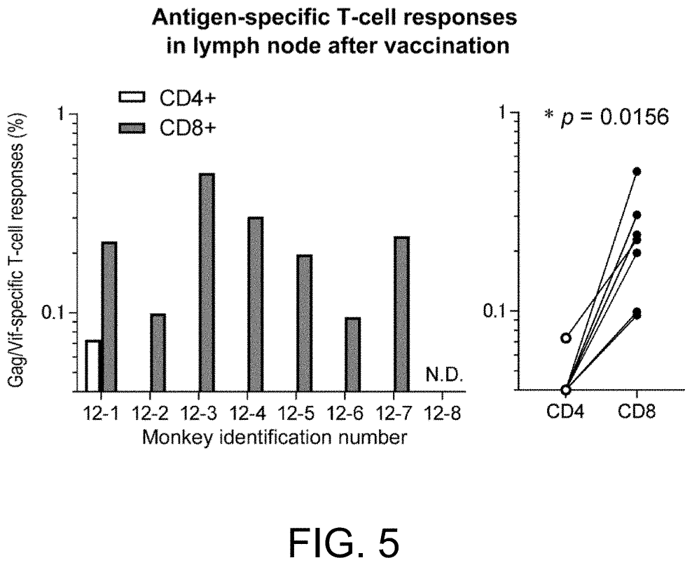

The result of analyzing antigen-specific T-cell responses in the post-vaccination lymph node showed that Gag/Vif-specific CD8-positive T-cell responses were also selectively induced in the lymph node, and Gag/Vif-specific CD4-positive T-cell responses were below the detection limit except for one animal (FIG. 5). As the lymph node is one of the major tissues in which HIV and SIV proliferate, the selective antigen-specific CD8-positive T-cell response in the lymph node may contribute to the replication control of HIV and SIV

### INDUSTRIAL APPLICABILITY

In antigen optimization studies aiming to induce effective CD8-positive T cells, the analyses of HIV-infected people and simian AIDS models have shown that CD8-positive T cell responses targeting Gag and Vif antigens are able to suppress virus replication potently. Meanwhile, based on the idea that antigen regions relatively conserved among various HIV strains may be CD8-positive T cell targets in which the selection of escape mutations is unlikely to occur, an antigen consisting of these conserved regions connected together has been designed (Letourneau S. et al., PLoS One. 2:e984, 2007). Moreover, an antigen in which regions including CD8-positive T cell targets associated with low viral loads (highly capable of suppressing HIV replication) are connected has also been designed (Mothe B. et al., J Transl Med. 9:208, 2011). These antigens all have Gag CA and Vif regions as the main regions. However, there has so far been no antigen designed from the viewpoint of inducing effective HIV antigen-specific CD8-positive T cells selectively while suppressing the induction of HIV antigen-specific CD4-positive T cells as much as possible. Therefore, the novelty, originality, and superiority of the present invention is extremely high. Moreover, the present antigen design theory is also applicable to the design of the above-mentioned antigens consisting of conserved regions connected together or potent HIV replication-suppressing CD8-positive T cell targets connected together. The present invention is expected to pave the way for a more effective vaccine therapy against AIDS.

1. A polypeptide comprising multiple peptides connected together, wherein each of the multiple peptides has an amino acid sequence of eight to twelve residues included in the amino acid sequence of an antigen protein.

2. The polypeptide of claim 1, wherein the eight- to twelve-residue peptides are connected in an order different from that in the antigen protein.

3. The polypeptide of claim 1 or 2, which does not substantially comprise a partial amino acid sequence of 13 or more consecutive residues in the antigen protein.

4. The polypeptide of any one of claims 1 to 3, wherein the amino acid sequences of the multiple peptides optionally comprise an overlap.

5. The polypeptide of claim 4, wherein the overlap consists of one to four residues.

6. The polypeptide of any one of claims 1 to 5, wherein each of the connection sites optionally comprises a spacer.

7. The polypeptide of claim 6, wherein the spacer consists of one to four amino acid residues.

8. The polypeptide of any one of claims 1 to 7, wherein at least 20 eight- to twelve-residue peptides are connected together.

9. A nucleic acid encoding the polypeptide of any one of claims 1 to 8.

10. A vector comprising the nucleic acid of claim 9.

11. The vector of claim 10, which is a Sendai virus vector.

12. A vaccine comprising the polypeptide of any one of claims 1 to 8, a nucleic acid encoding the polypeptide, or a vector comprising the nucleic acid.

13. The vaccine of claim 12, wherein the antigen protein is derived from an antigen protein of a human immunodeficiency virus.

14. A method for selectively inducing CD8-positive T cells specific for a target antigen, which comprises inoculating the vaccine of claim 12 or 13.

15. A method for producing the polypeptide of claim 1 or a nucleic acid encoding the polypeptide, which comprises:
(i) dividing an amino acid sequence encoding an antigen protein into amino acid sequences of eight to twelve residues, wherein the divided amino acid sequences may or may not overlap with one another;
(ii) connecting the divided amino acid sequences in such a way as not to become the same as the amino acid sequence of the antigen protein, wherein a spacer may or may not be inserted in each of the connection sites of the divided amino acid sequences; and
(iii) obtaining a polypeptide comprising an amino acid sequence resulting from step (ii) or a nucleic acid encoding the polypeptide.


SEQUENCE LISTING (54 sequences)

SEQ ID NO: 1 - protein, 60 aa, Artificial Sequence
  an artificially synthesized sequence
YPVQQIGGNYVHLPLSPRTLNAWVKLIEEKKFGAEVVPGFQALSEGCTPYDINQMLNCVG

SEQ ID NO: 2 - protein, 11 aa, Artificial Sequence
  an artificially synthesized sequence
YPVQQIGGNYV

SEQ ID NO: 3 - protein, 11 aa, Artificial Sequence
  an artificially synthesized sequence
HLPLSPRTLNA

SEQ ID NO: 4 - protein, 11 aa, Artificial Sequence
  an artificially synthesized sequence
WVKLIEEKKFG

SEQ ID NO: 5 - protein, 11 aa, Artificial Sequence
  an artificially synthesized sequence
AEVVPGFQALS

SEQ ID NO: 6 - protein, 11 aa, Artificial Sequence
  an artificially synthesized sequence
EGCTPYDINQM

SEQ ID NO: 7 - protein, 11 aa, Artificial Sequence
  an artificially synthesized sequence
LSPRTLNAWVK

SEQ ID NO: 8 - protein, 11 aa, Artificial Sequence
  an artificially synthesized sequence
EKKFGAEVVPG

SEQ ID NO: 9 - protein, 11 aa, Artificial Sequence
  an artificially synthesized sequence
LSEGCTPYDIN

SEQ ID NO: 10 - protein, 11 aa, Artificial Sequence
  an artificially synthesized sequence
NYVHLPLSPRT

SEQ ID NO: 11 - protein, 11 aa, Artificial Sequence
  an artificially synthesized sequence
PRTLNAWVKLI

SEQ ID NO: 12 - protein, 11 aa, Artificial Sequence
  an artificially synthesized sequence
KLIEEKKFGAE

SEQ ID NO: 13 - protein, 11 aa, Artificial Sequence
  an artificially synthesized sequence
GAEVVPGFQAL

SEQ ID NO: 14 - protein, 11 aa, Artificial Sequence
  an artificially synthesized sequence
QALSEGCTPYD

SEQ ID NO: 15 - protein, 11 aa, Artificial Sequence
  an artificially synthesized sequence
PYDINQMLNCV

SEQ ID NO: 16 - protein, 8 aa, Artificial Sequence
  an artificially synthesized sequence
NYVHLPLS

SEQ ID NO: 17 - protein, 10 aa, Artificial Sequence
  an artificially synthesized sequence
SPRTLNAWVK

SEQ ID NO: 18 - protein, 9 aa, Artificial Sequence
  an artificially synthesized sequence
EEKKFGAEV

SEQ ID NO: 19 - protein, 11 aa, Artificial Sequence
  an artificially synthesized sequence
EVVPGFQALSE

SEQ ID NO: 20 - protein, 10 aa, Artificial Sequence
  an artificially synthesized sequence
VYYDPSKDLI

SEQ ID NO: 21 - protein, 510 aa, Simian immunodeficiency virus
MGVRNSVLSGKKADELEKIRLRPNGKKKYMLKHVVWAANELDRFGLAESLLENKEGCQKILSVLAPLVPTGSENLKSLYNTVCVIWCIHAEEKVKHTEEAKQIVQRHLVVETGTTETMPKTSRPTAPSSGRGGNYPVQQIGGNYVHLPLSPRTLNAWVKLIEEKKFGAEVVPGFQALSEGCTPYDINQMLNCVGDHQAAMQIIRDIINEEAADWDLQHPQPAPQQGQLREPSGSDIAGTTSSVDEQIQWMYRQQNPIPVGNIYRRWIQLGLQKCVRMYNPTNILDVKQGPKEPFQSYVDRFYKSLRAEQTDAAVKNWMTQTLLIQNANPDCKLVLKGLGVNPTLEEMLTACQGVGGPGQKARLMAEALKEALAPVPIPFAAAQQRGPRKPIKCWNCGKEGHSARQCRAPRRQGCWKCGKMDHVMAKCPDRQAGFLGLGPWGKKPRNFPMAQVHQGLMPTAPPEDPAVDLLKNYMQLGKQQREKQRESREKPYKEVTEDLLHLNSLFGGDQ

SEQ ID NO: 22 - protein, 214 aa, Simian immunodeficiency virus
MEEEKRWIAVPTWRIPERLERWHSLIKYLKYKTKDLQKVCYVPHFKVGWAWWTCSRVIFPLQEGSHLEVQGYWHLTPEKGWLSTYAVRITWYSKNFWTDVTPNYADILLHSTYFPCFTAGEVRRAIRGEQLLSCCRFPRAHKYQVPSLQYLALKVVSDVRSQGENPTWKQWRRDNRRGLRMAKQNSRGDKQRGGKPPTKGANFPGLAKVLGILA

SEQ ID NO: 23 - protein, 674 aa, Artificial Sequence
  an artificially synthesized sequence
MAYPVQQIGGNYVAGAEVVPGFQALAAMQIIRDIINEAPSGSDIAGTTSAYRRWIQLGLQKAQSYVDRFYKSLAANPDCKLVLKGAQKARLMAEALKANYVHLPLSPRTAQALSEGCTPYDAINEEAADWDLQATTSSVDEQIQWALQKCVRMYNPTAKSLRAEQTDAAALKGLGVNPTLEAALKEALAPVPIAPRTLNAWVKLIAPYDINQMLNCVADLQHPQPAPQQAIQWMYRQQNPIANPTNILDVKQGADAAVKNWMTQTATLEEMLTACQGAVPIPFAAAQQRAKLIEEKKFGAEANCVGDHQAAMQAPQQGQLREPSGANPIPVGNIYRRAKQGPKEPFQSYATQTLLIQNANPACQGVGGPGQKAAQQRGPRKPIKCAMEEEKRWIAVPATKDLQKVCYVPASHLEVQGYWHLAWTDVTPNYADIAEQLLSCCRFPRASQGENPTWKQWAAVPTWRIPERLAYVPHFKVGWAWAWHLTPEKGWLSAADILLHSTYFPAFPRAHKYQVPSAKQWRRDNRRGLAERLERWHSLIKAWAWWTCSRVIFAWLSTYAVRITWAYFPCFTAGEVRAVPSLQYLALKVARGLRMAKQNSRALIKYLKYKTKDAVIFPLQEGSHLAITWYSKNFWTDAEVRRAIRGEQLALKVVSDVRSQGANSRGDKQRGGKA

SEQ ID NO: 24 - protein, 674 aa, Artificial Sequence
  an artificially synthesized sequence
MAPVQQIGGNYVHAAEVVPGFQALSAMQIIRDIINEEASGSDIAGTTSSARRWIQLGLQKCASYVDRFYKSLRANPDCKLVLKGLAKARLMAEALKEAYVHLPLSPRTLAALSEGCTPYDIANEEAADWDLQHATSSVDEQIQWMAQKCVRMYNPTNASLRAEQTDAAVAKGLGVNPTLEEALKEALAPVPIPARTLNAWVKLIEAYDINQMLNCVGALQHPQPAPQQGAQWMYRQQNPIPAPTNILDVKQGPAAAVKNWMTQTLALEEMLTACQGVAPIPFAAAQQRGALIEEKKFGAEVACVGDHQAAMQIAQQGQLREPSGSAPIPVGNIYRRWAQGPKEPFQSYVAQTLLIQNANPDAQGVGGPGQKARAQRGPRKPIKCWAEEEKRWIAVPTAKDLQKVCYVPHAHLEVQGYWHLTATDVTPNYADILAQLLSCCRFPRAAQGENPTWKQWRAVPTWRIPERLEAVPHFKVGWAWWAHLTPEKGWLSTADILLHSTYFPCAPRAHKYQVPSLAQWRRDNRRGLRARLERWHSLIKYAAWWTCSRVIFPALSTYAVRITWYAFPCFTAGEVRRAPSLQYLALKVVAGLRMAKQNSRGAIKYLKYKTKDLAIFPLQEGSHLEATWYSKNFWTDVAVRRAIRGEQLLAKVVSDVRSQGEASRGDKQRGGKPA

SEQ ID NO: 25 - protein, 674 aa, Artificial Sequence
  an artificially synthesized sequence
MAVQQIGGNYVHLAEVVPGFQALSEAQIIRDIINEEAAGSDIAGTTSSVARWIQLGLQKCVAYVDRFYKSLRAAPDCKLVLKGLGAARLMAEALKEAAVHLPLSPRTLNALSEGCTPYDINAEEAADWDLQHPASSVDEQIQWMYAKCVRMYNPTNIALRAEQTDAAVKAGLGVNPTLEEMAKEALAPVPIPFATLNAWVKLIEEADINQMLNCVGDAQHPQPAPQQGQAWMYRQQNPIPVATNILDVKQGPKAAVKNWMTQTLLAEEMLTACQGVGAIPFAAAQQRGPAIEEKKFGAEVVAVGDHQAAMQIIAQGQLREPSGSDAIPVGNIYRRWIAGPKEPFQSYVDATLLIQNANPDCAGVGGPGQKARLARGPRKPIKCWNAEEKRWIAVPTWADLQKVCYVPHFALEVQGYWHLTPADVTPNYADILLALLSCCRFPRAHAGENPTWKQWRRAPTWRIPERLERAPHFKVGWAWWTALTPEKGWLSTYAILLHSTYFPCFARAHKYQVPSLQAWRRDNRRGLRMALERWHSLIKYLAWWTCSRVIFPLASTYAVRITWYSAPCFTAGEVRRAASLQYLALKVVSALRMAKQNSRGDAKYLKYKTKDLQAFPLQEGSHLEVAWYSKNFWTDVTARRAIRGEQLLSAVVSDVRSQGENARGDKQRGGKPPA

SEQ ID NO: 26 - protein, 674 aa, Artificial Sequence
  an artificially synthesized sequence
MAQQIGGNYVHLPAVVPGFQALSEGAIIRDIINEEAAASDIAGTTSSVDAWIQLGLQKCVRAVDRFYKSLRAEADCKLVLKGLGVARLMAEALKEALAHLPLSPRTLNAASEGCTPYDINQAEAADWDLQHPQASVDEQIQWMYRACVRMYNPTNILARAEQTDAAVKNALGVNPTLEEMLAEALAPVPIPFAALNAWVKLIEEKAINQMLNCVGDHAHPQPAPQQGQLAMYRQQNPIPVGANILDVKQGPKEAVKNWMTQTLLIAEMLTACQGVGGAPFAAAQQRGPRAEEKKFGAEVVPAGDHQAAMQIIRAGQLREPSGSDIAPVGNIYRRWIQAPKEPFQSYVDRALLIQNANPDCKAVGGPGQKARLMAGPRKPIKCWNCAEKRWIAVPTWRALQKVCYVPHFKAEVQGYWHLTPEAVTPNYADILLHALSCCRFPRAHKAENPTWKQWRRDATWRIPERLERWAHFKVGWAWWTCATPEKGWLSTYAALLHSTYFPCFTAAHKYQVPSLQYARRDNRRGLRMAAERWHSLIKYLKAWTCSRVIFPLQATYAVRITWYSKACFTAGEVRRAIALQYLALKVVSDARMAKQNSRGDKAYLKYKTKDLQKAPLQEGSHLEVQAYSKNFWTDVTPARAIRGEQLLSCAVSDVRSQGENPAGDKQRGGKPPTA

SEQ ID NO: 27 - protein, 674 aa, Artificial Sequence
  an artificially synthesized sequence
MAQIGGNYVHLPLAVPGFQALSEGCAIRDIINEEAADADIAGTTSSVDEAIQLGLQKCVRMADRFYKSLRAEQACKLVLKGLGVNALMAEALKEALAALPLSPRTLNAWAEGCTPYDINQMAAADWDLQHPQPAVDEQIQWMYRQAVRMYNPTNILDAAEQTDAAVKNWAGVNPTLEEMLTAALAPVPIPFAAANAWVKLIEEKKANQMLNCVGDHQAPQPAPQQGQLRAYRQQNPIPVGNAILDVKQGPKEPAKNWMTQTLLIQAMLTACQGVGGPAFAAAQQRGPRKAEKKFGAEVVPGADHQAAMQIIRDAQLREPSGSDIAAVGNIYRRWIQLAKEPFQSYVDRFALIQNANPDCKLAGGPGQKARLMAAPRKPIKCWNCGAKRWIAVPTWRIAQKVCYVPHFKVAVQGYWHLTPEKATPNYADILLHSASCCRFPRAHKYANPTWKQWRRDNAWRIPERLERWHAFKVGWAWWTCSAPEKGWLSTYAVALHSTYFPCFTAAHKYQVPSLQYLARDNRRGLRMAKARWHSLIKYLKYATCSRVIFPLQEAYAVRITWYSKNAFTAGEVRRAIRAQYLALKVVSDVAMAKQNSRGDKQALKYKTKDLQKVALQEGSHLEVQGASKNFWTDVTPNAAIRGEQLLSCCASDVRSQGENPTADKQRGGKPPTKA

SEQ ID NO: 28 - protein, 674 aa, Artificial Sequence
  an artificially synthesized sequence
MAIGGNYVHLPLSAPGFQALSEGCTARDIINEEAADWAIAGTTSSVDEQAQLGLQKCVRMYARFYKSLRAEQTAKLVLKGLGVNPAMAEALKEALAPAPLSPRTLNAWVAGCTPYDINQMLAADWDLQHPQPAADEQIQWMYRQQARMYNPTNILDVAEQTDAAVKNWMAVNPTLEEMLTAALAPVPIPFAAAAAWVKLIEEKKFAQMLNCVGDHQAAQPAPQQGQLREARQQNPIPVGNIALDVKQGPKEPFANWMTQTLLIQNALTACQGVGGPGAAAAQQRGPRKPAKKFGAEVVPGFAHQAAMQIIRDIALREPSGSDIAGAGNIYRRWIQLGAEPFQSYVDRFYAIQNANPDCKLVAGPGQKARLMAEARKPIKCWNCGKARWIAVPTWRIPAKVCYVPHFKVGAQGYWHLTPEKGAPNYADILLHSTACCRFPRAHKYQAPTWKQWRRDNRARIPERLERWHSAKVGWAWWTCSRAEKGWLSTYAVRAHSTYFPCFTAGAKYQVPSLQYLAADNRRGLRMAKQAWHSLIKYLKYKACSRVIFPLQEGAAVRITWYSKNFATAGEVRRAIRGAYLALKVVSDVRAAKQNSRGDKQRAKYKTKDLQKVCAQEGSHLEVQGYAKNFWTDVTPNYAIRGEQLLSCCRADVRSQGENPTWAKQRGGKPPTKGA

SEQ ID NO: 29 - protein, 674 aa, Artificial Sequence
  an artificially synthesized sequence
MAGGNYVHLPLSPAGFQALSEGCTPADIINEEAADWDAAGTTSSVDEQIALGLQKCVRMYNAFYKSLRAEQTDALVLKGLGVNPTAAEALKEALAPVALSPRTLNAWVKACTPYDINQMLNADWDLQHPQPAPAEQIQWMYRQQNAMYNPTNILDVKAQTDAAVKNWMTANPTLEEMLTACAAPVPIPFAAAQAWVKLIEEKKFGAMLNCVGDHQAAAPAPQQGQLREPAQQNPIPVGNIYADVKQGPKEPFQAWMTQTLLIQNAATACQGVGGPGQAAAQQRGPRKPIAKFGAEVVPGFQAQAAMQIIRDIIAREPSGSDIAGTANIYRRWIQLGLAPFQSYVDRFYKAQNANPDCKLVLAPGQKARLMAEAAKPIKCWNCGKEAWIAVPTWRIPEAVCYVPHFKVGWAGYWHLTPEKGWANYADILLHSTYACRFPRAHKYQVATWKQWRRDNRRAIPERLERWHSLAVGWAWWTCSRVAKGWLSTYAVRIASTYFPCFTAGEAYQVPSLQYLALANRRGLRMAKQNAHSLIKYLKYKTASRVIFPLQEGSAVRITWYSKNFWAAGEVRRAIRGEALALKVVSDVRSAKQNSRGDKQRGAYKTKDLQKVCYAEGSHLEVQGYWANFWTDVTPNYAARGEQLLSCCRFAVRSQGENPTWKAQRGGKPPTKGAA

SEQ ID NO: 30 - protein, 674 aa, Artificial Sequence
  an artificially synthesized sequence
MAGNYVHLPLSPRAFQALSEGCTPYAIINEEAADWDLAGTTSSVDEQIQAGLQKCVRMYNPAYKSLRAEQTDAAVLKGLGVNPTLAEALKEALAPVPASPRTLNAWVKLATPYDINQMLNCAWDLQHPQPAPQAQIQWMYRQQNPAYNPTNILDVKQATDAAVKNWMTQAPTLEEMLTACQAPVPIPFAAAQQAVKLIEEKKFGAALNCVGDHQAAMAAPQQGQLREPSAQNPIPVGNIYRAVKQGPKEPFQSAMTQTLLIQNANAACQGVGGPGQKAAQQRGPRKPIKAFGAEVVPGFQAAAAMQIIRDIINAEPSGSDIAGTTAIYRRWIQLGLQAFQSYVDRFYKSANANPDCKLVLKAGQKARLMAEALAPIKCWNCGKEGAIAVPTWRIPERACYVPHFKVGWAAYWHLTPEKGWLAYADILLHSTYFARFPRAHKYQVPAWKQWRRDNRRGAPERLERWHSLIAGWAWWTCSRVIAGWLSTYAVRITATYFPCFTAGEVAQVPSLQYLALKARRGLRMAKQNSASLIKYLKYKTKARVIFPLQEGSHARITWYSKNFWTAGEVRRAIRGEQAALKVVSDVRSQAQNSRGDKQRGGAKTKDLQKVCYVAGSHLEVQGYWHAFWTDVTPNYADAGEQLLSCCRFPARSQGENPTWKQARGGKPPTKGANA

SEQ ID NO: 31 - DNA, 2025 bp, Artificial Sequence
  an artificially synthesized sequence
atggcctaccctgtgcagcagatcggaggcaattacgtggccggagctgaggtggtgccaggatttcaggctctggccgctatgcagatcatcagggacatcatcaatgaggccccaagcggatctgacatcgccggcaccacctctgcctataggagatggatccagctgggcctgcaaaaggcccagagctacgtggataggttttataaatccctggctgccaatcctgactgcaaactggtgctgaaaggagctcagaaagctcggctgatggctgaggccctcaaggctaattatgtgcatctgccactgtctcccaggacagcccaggccctgtccgagggatgcaccccttatgacgccatcaacgaggaggccgctgactgggatctccaggctaccacatcttccgtggatgagcagatccaatgggccctgcagaagtgcgtgagaatgtacaaccctacagctaaatctctgagagctgagcagaccgacgctgctgccctgaagggactgggcgtgaatccaacactcgaagctgccctgaaggaggctctggctcctgtgcccatcgctccccgcacactgaacgcctgggtgaagctcatcgccccttacgacatcaaccagatgctgaattgtgtggctgatctgcagcatccacagccagctcctcaacaggccatccagtggatgtaccgccagcagaaccctatcgccaaccccacaaatatcctggacgtgaaacaaggcgccgacgccgctgtgaagaactggatgacccaaacagccacactggaggagatgctgaccgcttgtcaaggagccgtgccaatccccttcgccgctgcccagcaaagggccaagctgatcgaggagaaaaagttcggagccgaggccaattgcgtgggggatcaccaggccgccatgcaagcccctcagcagggacagctgagggagccatccggagctaacccaatccccgtgggaaatatctatagaagagccaaacagggccctaaggagccattccagtcctacgccacccagacactgctgatccagaacgccaaccctgcctgtcagggagtgggaggacctggacagaaggctgctcagcagaggggaccaaggaaacctatcaaatgcgccatggaggaggagaaaaggtggatcgccgtcccagccaccaaggacctgcagaaagtgtgctatgtgcccgcctcccacctggaggtgcagggatactggcacctggcctggaccgacgtgacacccaattacgctgacatcgctgagcagctgctgtcctgctgtaggtttcccagagccagccagggcgagaacccaacctggaagcaatgggcagccgtgccaacctggagaatccccgagaggctggcctacgtgccacatttcaaagtggggtgggcatgggcctggcatctgacccctgagaaaggatggctcagcgccgctgatatcctgctgcactctacctacttccccgcctttcctagagcccataaataccaggtgcccagcgccaagcagtggcggcgcgacaataggagaggcctggccgagcggctggagaggtggcactccctgattaaggcctgggcttggtggacatgttctagagtcatcttcgcctggctgagcacctacgccgtgagaatcacctgggcctatttcccttgctttacagccggcgaggtcagggccgtgccaagcctgcagtatctggctctgaaagtggccagaggactgcgcatggccaaacagaactctagggctctgatcaagtacctgaagtataaaaccaaagacgccgtgatctttccactgcaggagggatctcacctcgccatcacatggtattctaagaacttctggacagacgctgaggtgaggagagctatcaggggggagcaactggccctgaaggtggtgtccgatgtgcggagccaaggcgctaactccaggggcgataagcagaggggaggcaaagcctga

SEQ ID NO: 32 - DNA, 2025 bp, Artificial Sequence
  an artificially synthesized sequence
atggcccctgtgcagcagatcggaggcaattatgtccatgccgctgaggtggtgccaggatttcaggccctctccgccatgcagatcatcagggacatcatcaacgaagaggccagcggatctgacatcgccggcaccacatcctctgccaggagatggatccagctgggcctgcagaaatgcgccagctacgtggataggttttataaatctctcagagccaatcctgactgcaaactggtgctgaagggcctggccaaagctcggctgatggctgaggccctgaaagaggcttatgtgcatctgccactgtctccccgcactctggccgccctgtccgagggatgcaccccttacgatatcgccaacgaggaggccgctgactgggatctgcaacatgctacatcttccgtggatgagcagatccaatggatggcccagaagtgcgtgagaatgtacaaccccactaatgcctctctgagagctgagcagaccgacgccgcagtggccaagggactgggcgtgaatccaacactggaagaagccctgaaggaggctctggctcctgtgccaattcccgctcgcacactgaacgcctgggtgaagctcatcgaggcttacgacatcaaccagatgctgaattgcgtcggggccctgcagcatccacagccagctcctcagcaaggagctcagtggatgtaccgccagcagaacccaattcccgcccccacaaatatcctggacgtgaaacagggacctgccgccgctgtgaagaactggatgacccagactctggctctggaggagatgctgaccgcttgtcagggcgtggccccaatccccttcgccgctgcccagcagagaggagccctgatcgaggagaaaaagttcggagctgaagtcgcctgcgtgggggatcaccaggccgctatgcaaatcgcccagcagggacagctgagggagccaagcggctctgctccaatccccgtgggaaatatctataggaggtgggcccagggccctaaggagccattccagagctatgtggctcagacactgctgatccagaacgccaatcccgacgcccagggagtgggaggacctggacagaaagcacgggcccagaggggaccaaggaaacctatcaagtgttgggctgaggaggagaaaaggtggatcgccgtgcctaccgctaaggacctgcagaaagtgtgctacgtcccacatgcccacctggaggtgcagggatactggcatctcaccgccaccgacgtgacacccaattacgctgatattctcgcccagctgctgtcctgctgtaggtttcctagggccgctcagggcgagaacccaacctggaagcaatggagggccgtgccaacctggagaatccccgagaggctggaagccgtgccacatttcaaagtggggtgggcctggtgggctcatctgacccctgagaaaggatggctgtccaccgcagatatcctgctgcactctacctatttcccatgcgcccctagagcccataaataccaggtgccatccctggcccagtggcggcgcgacaataggagaggactccgcgcccggctggagaggtggcactccctgatcaaatacgccgcttggtggacatgttctagagtgatcttcccagccctgagcacctacgccgtgagaatcacctggtacgccttcccttgctttacagccggcgaggtgcggagagccccaagcctgcagtatctggctctgaaggtcgtggccggactgcgcatggccaaacagaactccagaggcgccatcaagtacctgaagtataaaaccaaggatctggccatctttccactgcaggagggatcccatctggaagctacatggtattctaagaacttctggacagacgtcgccgtgaggagagctatcaggggggagcagctcctggctaaggtggtgtccgatgtgcggagccagggagaggcttccaggggcgataagcagaggggagggaagcccgcctga

SEQ ID NO: 33 - DNA, 2025 bp, Artificial Sequence
  an artificially synthesized sequence
atggccgtgcagcagatcggaggcaattatgtgcacctggccgaggtggtgccaggatttcaggccctgtctgaggcccagatcatcagggacatcatcaacgaggaagccgccggatctgacatcgccggcaccacatctagcgtggccagatggatccagctgggcctgcagaagtgtgtggcttacgtggataggttttataaatctctgagggctgcccctgactgcaaactggtgctgaagggactcggcgccgctcggctgatggctgaggccctgaaggaagctgctgtgcatctgccactgtctccccgcacactcaacgccctgtccgagggatgcaccccttacgacattaacgctgaggaggccgctgactgggatctgcagcacccagcctcttccgtggatgagcagatccagtggatgtatgccaagtgcgtgagaatgtacaaccccacaaacatcgccctgagagctgagcagaccgacgccgctgtcaaggctggactgggcgtgaatccaacactggaggaaatggccaaggaggctctggctcctgtgccaatcccattcgccacactgaacgcctgggtgaagctgattgaggaagccgacatcaaccagatgctgaattgcgtgggcgatgctcagcatccacagccagctcctcagcaggggcaggcctggatgtaccgccagcagaacccaatccctgtggccacaaatatcctggacgtgaaacagggccccaaagccgctgtgaagaactggatgacccagactctgctcgccgaggagatgctgaccgcttgtcagggagtcggagccatccccttcgccgctgcccagcagaggggcccagccatcgaggagaaaaagttcggagctgaggtcgtggctgtgggggatcaccaggccgctatgcagattatcgctcagggacagctgagggagccaagcggatccgacgccatccccgtgggaaatatctataggaggtggattgccggccctaaggagccattccagagctacgtcgatgccacactgctgatccagaacgccaatcctgattgcgccggagtgggaggacctggacagaaagctaggctggctaggggaccaaggaaacctatcaagtgttggaatgccgaggagaaaaggtggatcgccgtgccaacatgggccgacctgcagaaagtgtgctacgtgccacacttcgctctggaggtgcagggatactggcatctgacacctgccgacgtgacacccaattacgctgatatcctcctggcactgctgtcctgctgtaggtttcctagagctcatgccggcgagaacccaacctggaagcagtggaggcgcgccccaacctggagaatccccgagcggctggaaagggctccacatttcaaagtggggtgggcttggtggaccgccctgacccctgagaaaggatggctgagcacatacgccatcctgctgcactctacctatttcccttgttttgccagagcccataaataccaggtgccaagcctccaggcttggcggcgcgacaataggagaggactgcggatggctctggagaggtggcactccctgatcaagtatctcgcctggtggacatgttctagagtgatctttcctctggctagcacctacgccgtgagaatcacatggtactctgccccttgctttacagccggcgaggtgaggagggctgcaagcctgcagtatctggctctgaaggtggtctccgccctgcgcatggccaaacagaactccaggggagatgccaagtacctgaagtataaaaccaaggacctccaggcatttccactgcaggagggatcccacctggaagtggcctggtattctaagaacttctggaccgacgtcacagccaggagagctatcaggggggagcagctgctctccgccgtggtgtccgatgtgcggagccagggcgaaaacgccaggggcgataagcagaggggagggaaacctcctgcttga

SEQ ID NO: 34 - DNA, 2025 bp, Artificial Sequence
  an artificially synthesized sequence
atggcccagcagatcggaggcaattatgtgcatctcccagccgtggtgccaggatttcaggccctgtccgaaggagccatcatcagggacatcatcaacgaggaggctgctgcctctgacatcgccggcaccacatcttccgtcgatgcctggatccagctgggcctgcagaagtgtgtgagggccgtggataggttttataaatctctgagagccgaggccgactgcaaactggtgctgaagggactgggagtggcccggctgatggctgaggccctgaaggaggccctggcccatctgccactgtctccccgcacactgaatgccgcctccgagggatgcaccccttacgacatcaatcaggccgaggccgctgactgggatctgcagcatccccaggcttccgtggatgagcagatccagtggatgtatcgcgcctgcgtgagaatgtacaaccccacaaatattctggctagagctgagcagaccgacgccgctgtgaaaaacgccctgggcgtgaatccaacactggaggaaatgctcgccgaggctctggctcctgtgccaatcccctttgccgctctgaacgcctgggtgaagctgatcgaggaaaaagccatcaaccagatgctgaattgcgtgggggaccacgcccatccacagccagctcctcagcagggacaactggcaatgtaccgccagcagaacccaatccccgtcggggccaatatcctggacgtgaaacagggccctaaagaggctgtgaagaactggatgacccagacactgctcatcgccgagatgctgaccgcttgtcagggagtgggcggagctcccttcgccgctgcccagcagaggggacccagggctgaggagaaaaagttcggagctgaggtggtcccagctggggatcaccaggccgctatgcagatcattcgggccggacagctgagggagccaagcggatctgatatcgctcccgtgggaaatatctataggagatggattcaggctcctaaggagccattccagagctacgtggacagggccctgctgatccagaacgccaatcctgactgtaaagccgtgggaggacctggacagaaagctcggctcatggccggaccaaggaaacctatcaagtgctggaattgtgccgagaaaaggtggatcgccgtgccaacatggagggctctgcagaaagtgtgctacgtgccacattttaaagctgaggtgcagggatactggcatctgaccccagaggccgtgacacccaattacgctgatatcctgctccacgctctgtcctgctgtaggtttcctagagcccacaaggccgagaacccaacctggaagcagtggcggcgggacgccacctggagaatccccgagcggctggagagatgggcccatttcaaagtggggtgggcttggtggacctgtgccacccctgagaaaggatggctgagcacctatgctgctctgctgcactctacctatttcccttgcttcacagccgcccataaataccaggtgccaagcctgcaatatgcccggcgcgacaataggagaggactgcggatggcagccgagaggtggcactccctgatcaagtacctcaaggcttggacatgttctagagtgatctttcctctgcaagccacctacgccgtgagaatcacatggtattccaaggcctgctttacagccggcgaggtgaggagagccatcgccctgcagtatctggctctgaaggtggtgtctgatgcccgcatggccaaacagaactccaggggcgacaaggcatacctgaagtataaaaccaaggacctgcaaaaagccccactgcaggagggatcccacctggaggtccaggcctattctaagaacttctggaccgacgtgactcccgccagagctatcaggggggagcagctgctgtcttgcgccgtgtccgatgtgcggagccagggcgagaatccagccggcgataagcagaggggagggaaacccccaacagcctga

SEQ ID NO: 35 - DNA, 2025 bp, Artificial Sequence
  an artificially synthesized sequence
atggcccagatcggaggcaattatgtgcatctgcctctggccgtgccaggatttcaggccctgtccgaggggtgcgccatcagggacatcatcaacgaggaggccgccgacgccgacatcgccggcaccacatcttccgtggacgaggccatccagctgggcctgcagaagtgcgtgaggatggctgataggttttataaatctctgagagctgaacaggcctgcaaactggtgctgaagggactgggcgtcaatgccctgatggctgaggccctgaaggaggctctcgctgccctgccactgtctccccgcacactgaacgcttgggccgagggatgcaccccttacgacatcaaccaaatggccgccgctgactgggatctgcagcatccccagcctgccgtggatgagcagatccagtggatgtaccggcaggctgtgagaatgtacaaccccacaaatatcctcgacgctgctgagcagaccgacgccgctgtgaagaattgggctggcgtgaatccaacactggaggagatgctcaccgccgctctggctcctgtgccaatccccttcgctgctgctaacgcctgggtgaagctgatcgaggagaagaaagccaaccagatgctgaattgcgtgggggatcatcaagccccacagccagctcctcagcagggacagctcagggcctaccgccagcagaacccaatccccgtgggcaatgctatcctggacgtgaaacagggccctaaggaaccagccaagaactggatgacccagacactgctgattcaggccatgctgaccgcttgtcagggagtgggaggccctgccttcgccgctgcccagcagaggggaccacggaaggccgagaaaaagttcggagctgaggtggtgcctggagccgatcaccaggccgctatgcagatcatcagagacgcccagctgagggagccaagcggatctgacattgccgccgtgggaaatatctataggagatggatccaactggctaaggagccattccagagctacgtggatagatttgccctgatccagaacgccaatcctgactgcaagctggcaggaggacctggacagaaagctcggctcatggccgctccaaggaaacctatcaagtgctggaactgcggcgccaaaaggtggatcgccgtgccaacctggaggatcgcccagaaagtgtgctacgtgccacatttcaaggtggccgtgcagggatactggcatctgacccctgaaaaagctacacccaattacgctgatatcctgctgcattctgcctcctgctgtaggtttcctagagcccataagtacgccaacccaacctggaagcagtggcggcgcgataatgcatggagaatccccgagcggctggagagatggcatgccttcaaagtggggtgggcttggtggacatgctctgctcctgagaaaggatggctgagcacctacgctgtggctctgcactctacctatttcccttgctttaccgccgcccataaataccaggtgccaagcctgcagtacctggcccgcgacaataggagaggactgcgcatggctaaagcaaggtggcactccctgatcaagtacctgaaatatgccacatgttctagagtgatctttccactccaggaagcctacgccgtgagaatcacatggtattctaaaaacgcctttacagccggcgaggtgaggagagctattagggcccagtatctggctctgaaggtggtgtccgacgtggccatggccaaacagaactccaggggcgataaacaggctctgaagtataaaaccaaggacctgcagaaggtggctctgcaggagggatcccacctggaggtgcaaggagcttctaagaacttctggaccgacgtgacacctaatgccgctatcaggggggagcagctgctgtcctgttgtgcctccgatgtgcggagccagggcgagaaccctaccgctgataagcagaggggagggaaaccccctaccaaggcctga

SEQ ID NO: 36 - DNA, 2025 bp, Artificial Sequence
  an artificially synthesized sequence
atggccatcggaggcaattatgtgcatctgccactctctgccccaggatttcaggccctgtccgagggatgtacagccagggacatcatcaacgaggaggccgctgattgggccatcgccggcaccacatcttccgtggatgaacaggcccagctgggcctgcagaagtgcgtgaggatgtatgccaggttttataaatctctgagagctgagcaaaccgctaaactggtgctgaagggactgggcgtgaacccagccatggctgaggccctgaaggaggctctggcacctgccccactgtctccccgcacactgaacgcttgggtcgccggatgcaccccttacgacatcaaccaaatgctcgccgctgactgggatctgcagcatccacagcctgctgccgatgagcagatccagtggatgtaccgccaacaggctagaatgtacaaccccacaaatatcctggatgtggccgagcagaccgacgccgctgtgaagaattggatggctgtgaatccaacactggaggagatgctgacagctgctctggctcctgtgccaatccccttcgccgcagccgccgcctgggtgaagctgatcgaggagaaaaaattcgcccagatgctgaattgcgtgggggatcaccaagccgctcagccagctcctcagcagggacagctgagagaggcccgccagcagaacccaatccccgtgggaaacatcgccctggacgtgaaacagggccctaaggagcccttcgctaactggatgacccagacactgctgattcagaatgccctgaccgcttgtcagggagtgggaggaccaggagccgccgctgcccagcagaggggaccaaggaagcctgctaaaaagttcggagctgaggtggtgccaggctttgcccaccaggccgctatgcagatcatcagggatatcgctctgagggagccaagcggatctgacatcgctggcgccggaaatatctataggagatggatccagctcggcgctgagccattccagagctacgtggataggttctatgccatccagaacgccaatcctgactgcaaactcgtggctggacctggacagaaagctcggctgatggccgaggctaggaaacctatcaagtgctggaactgtggaaaggccaggtggatcgccgtgccaacctggaggattcccgccaaagtgtgctacgtgccacatttcaaagtcggggcccagggatactggcatctgacccctgagaagggagctcccaattacgctgatatcctgctgcactccaccgcttgctgtaggtttcctagagcccataaatatcaggcaccaacctggaagcagtggcggcgcgacaacagggccagaatccccgagcggctggagaggtggcattccgccaaagtggggtgggcttggtggacatgttccagagccgagaaaggatggctgagcacctacgccgtcagagctcactctacctatttcccttgctttacagctggcgctaaataccaggtgccaagcctgcagtatctcgctgctgacaataggagaggactgcgcatggccaagcaggcttggcactccctgatcaagtacctgaagtacaaagcctgttctagagtgatctttccactgcaggaaggagctgccgtgagaatcacatggtattctaagaatttcgccacagccggcgaggtgaggagagctatcagaggggcctatctggctctgaaggtggtgtccgatgtccgggccgccaaacagaactccaggggcgataagcaaagggctaagtataaaaccaaggacctgcagaaagtctgcgcccaggagggatcccacctggaggtgcaggggtacgccaagaacttctggaccgacgtgacacccaactacgctatcaggggggagcagctgctgtcctgctgcagggctgatgtgcggagccagggcgagaacccaacatgggccaagcagaggggagggaaaccccctacaaaaggcgcctga

SEQ ID NO: 37 - DNA, 2025 bp, Artificial Sequence
  an artificially synthesized sequence
atggccggaggcaattatgtgcatctgccactgtcccccgccggatttcaggccctgtccgagggatgcacacctgccgacatcatcaacgaggaggccgctgattgggacgccgccggcaccacatcttccgtggatgagcaaatcgccctgggcctgcagaagtgcgtgagaatgtataacgccttttataaatctctgagagctgagcagactgacgctctggtgctgaagggactgggcgtgaatcccacagccgctgaggccctgaaggaggctctggctccagtggccctgtctccccgcacactgaacgcctgggtcaaggcctgcaccccttacgacatcaaccagatgctcaatgccgactgggatctgcagcatccacagccagcccctgctgagcagatccagtggatgtaccgccagcaaaacgctatgtacaaccccacaaatatcctggacgtcaaagcccagaccgacgccgctgtgaagaactggatgactgccaatccaacactggaggagatgctgaccgcctgtgccgctcctgtgccaatccccttcgccgctgctcaggcctgggtgaagctgatcgaggagaaaaagtttggagccatgctgaattgcgtgggggatcaccaggcagctgccccagctcctcagcagggacagctgagggaaccagcccagcagaacccaatccccgtgggaaatatttatgccgacgtgaaacagggccctaaggagccatttcaggcttggatgacccagacactgctgatccagaatgccgccaccgcttgtcagggagtgggaggacctggccaggccgctgcccagcagaggggaccaaggaaacccattgccaagttcggagctgaggtggtgccaggattccaggctcaggccgctatgcagatcatcagggacattatcgctagggagccaagcggatctgacatcgccgggaccgccaatatctataggagatggatccagctgggactggccccattccagagctacgtggataggttttacaaagctcagaacgccaatcctgactgcaaactggtcctggctcctggacagaaagctcggctgatggctgaagccgctaaacctatcaagtgctggaactgtggcaaagaggcctggatcgccgtgccaacctggagaatccctgaggctgtgtgctacgtgccacatttcaaagtgggatgggccggatactggcatctgacccctgagaaagggtgggctaattacgctgatatcctgctgcactctacatatgcttgtaggtttcctagagcccataaatatcaggtcgccacctggaagcagtggcggcgcgacaatagaagagccatccccgagcggctggagaggtggcacagcctggcagtggggtgggcttggtggacatgttctagggtggccaaaggatggctgagcacctacgccgtgcggatcgcctctacctatttcccttgctttacagccggggaggcttaccaggtgccaagcctgcagtatctggccctcgccaataggagaggactgcgcatggccaagcagaatgcccactccctgatcaagtacctgaagtataagaccgcttctagagtgatctttccactgcaggagggctccgccgtgagaatcacatggtattctaagaacttttgggctgccggcgaggtgaggagagctatcaggggagaggcactggctctgaaggtggtgtccgatgtgaggagcgccaaacagaactccaggggcgataagcagagaggagcctataaaaccaaggacctgcagaaagtgtgttacgccgagggatcccacctggaggtgcagggatattgggccaacttctggaccgacgtgacacccaactacgcagccaggggggagcagctgctgtcctgctgtagatttgccgtgcggagccagggcgagaacccaacatggaaagctcagaggggagggaaaccccctacaaagggcgccgcctga

SEQ ID NO: 38 - DNA, 2025 bp, Artificial Sequence
  an artificially synthesized sequence
atggccggcaattatgtgcatctgccactgtctcctcgcgcctttcaggccctgtccgagggatgcaccccatacgccatcatcaacgaggaggccgctgactgggacctggccggcaccacatcttccgtggatgagcagattcaggccggcctgcagaagtgcgtgagaatgtacaatcccgcctataaatctctgagagctgagcagaccgatgctgccgtgctgaagggactgggcgtgaatccaaccctggctgaggccctgaaggaggctctggctcctgtcccagcttctccccgcacactgaacgcctgggtgaaactggccaccccttacgacatcaaccagatgctgaactgcgcctgggatctgcagcatccacagccagctccccaggcccagatccagtggatgtaccgccagcagaatccagcttacaaccccacaaatatcctggacgtgaagcaggccaccgacgccgctgtgaagaactggatgacacaggctccaacactggaggagatgctgaccgcttgccaagcccctgtgccaatccccttcgccgctgcccaacaggcagtgaagctgatcgaggagaaaaagttcggcgctgccctgaattgcgtgggggatcaccaggccgccatggccgctcctcagcagggacagctgagggagcccagcgctcagaacccaatccccgtgggaaatatctacagggctgtgaaacagggccctaaggagccatttcagtccgccatgacccagacactgctgatccagaacgctaatgccgcttgtcagggagtgggaggacctggccagaaggccgcccagcagaggggaccaaggaaacctattaaagccttcggagctgaggtggtgccaggatttcaagccgctgccgctatgcagatcatcagggacatcattaacgccgagccaagcggatctgacatcgccggcactacagccatctataggagatggatccagctgggcctccaggccttccagagctacgtggataggttttataagtctgccaacgccaatcctgactgcaaactggtgctcaaggctggacagaaagctcggctgatggctgaggctctcgctcctatcaagtgctggaactgtggcaaggaaggggccatcgccgtgccaacctggagaatccctgagagggcctgctacgtgccacatttcaaagtgggatgggccgcctactggcatctgacccctgagaaagggtggctcgcctacgctgatatcctgctgcactctacctacttcgccaggtttcctagagcccataaataccaggtcccagcctggaagcagtggcggcgcgacaataggaggggagcccccgagcggctggagaggtggcacagcctgattgccgggtgggcttggtggacatgttctagagtcatcgccggatggctgagcacctacgccgtgagaattacagctacctatttcccttgctttacagccggcgaagtggcccaggtgccaagcctgcagtatctggctctcaaggcaaggagaggactgcgcatggccaaacagaatagcgcctccctgatcaagtacctgaagtataaaacaaaggccagagtgatctttccactgcaggagggaagccacgccagaatcacatggtattctaagaacttttggacagccggcgaggtgaggagagctatcaggggggaacaagccgctctgaaggtggtgtccgatgtgcggtcccaggcacagaactccaggggcgataagcagaggggcggggctaaaaccaaggacctgcagaaagtgtgctatgtggctggatcccacctggaggtgcagggatattggcacgctttctggaccgacgtgacacccaattacgccgatgccggggagcagctgctgtcctgctgtaggttccctgcccggagccagggcgagaacccaacctggaaacaggccaggggagggaaaccccctacaaagggagctaacgcttga

SEQ ID NO: 39 - DNA, 42 bp, Artificial Sequence
  an artificially synthesized sequence
atatgcggccgcgacgccaccatggcctaccctgtgcagcag

SEQ ID NO: 40 - DNA, 68 bp, Artificial Sequence
  an artificially synthesized sequence
atatgcggccgcgatgaactttcaccctaagtttttcttactacggtcaggctttgcctcccctctgc

SEQ ID NO: 41 - DNA, 43 bp, Artificial Sequence
  an artificially synthesized sequence
atatgcggccgcgacgccaccatggcccctgtgcagcagatcg

SEQ ID NO: 42 - DNA, 68 bp, Artificial Sequence
  an artificially synthesized sequence
atatgcggccgcgatgaactttcaccctaagtttttcttactacggtcaggcgggcttccctcccctc

SEQ ID NO: 43 - DNA, 43 bp, Artificial Sequence
  an artificially synthesized sequence
atatgcggccgcgacgccaccatggccgtgcagcagatcggag

SEQ ID NO: 44 - DNA, 69 bp, Artificial Sequence
  an artificially synthesized sequence
atatgcggccgcgatgaactttcaccctaagtttttcttactacggtcaagcaggaggtttccctcccc

SEQ ID NO: 45 - DNA, 42 bp, Artificial Sequence
  an artificially synthesized sequence
atatgcggccgcgacgccaccatggcccagcagatcggaggc

SEQ ID NO: 46 - DNA, 69 bp, Artificial Sequence
  an artificially synthesized sequence
atatgcggccgcgatgaactttcaccctaagtttttcttactacggtcaggctgttgggggtttccctc

SEQ ID NO: 47 - DNA, 46 bp, Artificial Sequence
  an artificially synthesized sequence
atatgcggccgcgacgccaccatggcccagatcggaggcaattatg

SEQ ID NO: 48 - DNA, 69 bp, Artificial Sequence
  an artificially synthesized sequence
atatgcggccgcgatgaactttcaccctaagtttttcttactacggtcaggccttggtagggggtttcc

SEQ ID NO: 49 - DNA, 43 bp, Artificial Sequence
  an artificially synthesized sequence
atatgcggccgcgacgccaccatggccatcggaggcaattatg

SEQ ID NO: 50 - DNA, 67 bp, Artificial Sequence
  an artificially synthesized sequence
atatgcggccgcgatgaactttcaccctaagtttttcttactacggtcaggcgccttttgtaggggg

SEQ ID NO: 51 - DNA, 42 bp, Artificial Sequence
  an artificially synthesized sequence
atatgcggccgcgacgccaccatggccggaggcaattatgtg

SEQ ID NO: 52 - DNA, 69 bp, Artificial Sequence
  an artificially synthesized sequence
atatgcggccgcgatgaactttcaccctaagtttttcttactacggtcaggcggcgccctttgtagggg

SEQ ID NO: 53 - DNA, 42 bp, Artificial Sequence; identical to SEQ ID NO: 51
  an artificially synthesized sequence

SEQ ID NO: 54 - DNA, 69 bp, Artificial Sequence; identical to SEQ ID NO: 52
  an artificially synthesized sequence